(19) United States
(12) Patent Application Publication
(10) Pub. No.: US 2007/0134814 A1
(43) Pub. Date: Jun. 14, 2007

(54) Methods and compositions for the detection of calcifying nano-particles, identification and quantification of associated proteins thereon, and correlation to disease

(76) Inventors: E. Olavi Kajander, Lakeland, FL (US); Katja M. Aho, Siilinjarvi (FI); Neva Ciftcioglu, League City, TX (US)

Correspondence Address: NEEDLE & ROSENBERG, P.C., SUITE 1000, 999 PEACHTREE STREET, ATLANTA, GA, 30309-3915, US

(21) Appl. No.: 11/299,328

(22) Filed: Dec. 9, 2005

(51) Int. Cl. G01N 33/551

(52) U.S. Cl. 436/524

(57) ABSTRACT

Disclosed are methods and compositions for detecting, analyzing and assessing the significance of calcifying nano-particles. The disclosed methods and compositions generally involve detecting one or more proteins present on a calcifying nano-particle. It has been discovered that particular proteins become associated with calcifying nano-particles. This association provides a means for detecting, classifying, analyzing, categorizing, and assessing calcifying nano-particles. Detecting particular proteins while associated with a calcifying nano-particle can be used to indicate the presence and type of calcifying nano-particle, which can be used to indicate the presence of, or disposition to, diseases or conditions. Multiple proteins on a calcifying particle can be detected. The presence or absence of particular proteins and the pattern of the presence and absence of particular proteins can be used to indicate the presence and type of calcifying nano-particle.

### FIELD OF THE INVENTION

The disclosed invention is generally in the field of calcification and calcifying bodies and specifically in the area of calcifying nano-particles. For example, the present invention discloses methods and compositions for the identification of calcifying nano-particles and protein/calcifying nanoparticles complexes and the correlation of said particles to various diseases.

### BACKGROUND OF THE INVENTION

Calcifying nano-particles (CNPs) are approximately 200 nm in size and appear to multiply in the biological mode, meaning their growth curve has the same characteristics as that of a life form, i.e., certain doubling time (typically around 3 days), plus a lag, a logarithmic, a stationary and even a death phase. The particles are passageable apparently indefinitely in cell culture media (Kajander and [symbol] iftçioglu, Proc. Natl. Acad. Sci. USA 95, 8274 (1998)). The main structural component identified, without question, is bonelike apatite (Kajander and [symbol] iftçioglu, Proc. Natl. Acad. Sci. USA 95, 8274 (1998); Miller et al., Am. J. Physiol. Heart Circ. Physiol. 287, H1115 (2004); Cisar et al., Proc. Natl. Acad. Sci. USA. 97, 11511 (2000); Vali et al, Geochim. Cosmochim. Acta 65, 63 (2001); [symbol] iftçioglu et al., Kidney Int. 67, 483 (2005)). CNPs have been isolated from kidney stones ( [symbol] iftçioglu et al., Kidney Int. 56, 1893 (1999); Khullar et al., Urol. Res. 32, 190 (2004)), gall stones (Wen et al., Chin Med. J. 118, 421 (2005)), calcific cancer (Sedivy and Battistutti, APMIS 111, 951 (2003); Hudelist et al., Histopathology 45, 633 (2004)) and pathological calcifications (Miller et al., Am. J. Physiol. Heart Circ. Physiol. 287, H1115 (2004)). CNPs have been clearly differentiated from known biological entities: eubacteria, archaea, virus, prions and eukaryotes (Aho and Kajander, J. Clin. Microbiol. 41, 3460 (2003)).

CNPs have been shown to form mineral calcium or hydroxy apatite coatings on their surfaces. The hydroxy apatite surface acts an a mineral calcium substrate for the binding of calcium binding proteins (CaBP). Proteins that associate with the CNP Hydroxy apatite complex (CNP/HA complex) may undergo a conformational change. Subsequently, the CNP/HA CaBP complex may attract or bind proteins that have an affinity to the aforementioned bound CaBPs. Neoeopitope formation is causal for multiple binding by host proteins. Crosslink formation is causal for multiple binding of host protein and stabilizes the structure so that it is stable and can withstand washing steps, for example, detergents, freeze thawing, etc., step involved in assays and storage functions.

Copending application Ser. Nos. 11/102,798, 11/180,921, and 11/182,076 disclose methods and compositions for the treatment of CNPs and are incorporated by reference herein. Commonly assigned patents U.S. Pat. No. 6,706,290 (Eradication of Nanobacteria) and U.S. Pat. No. 5,135,851 (Culture and Detection Methods for the Sterile Filterable autonomously replicating biological particles) are incorporated by reference herein.

### BRIEF SUMMARY OF THE INVENTION

Disclosed are methods and compositions for detecting, analyzing and assessing the significance of calcifying nano-particles. The disclosed methods and compositions generally involve detecting one or more proteins present on a calcifying nano-particle. It has been discovered that particular proteins become associated with calcifying nano-particles. This association provides a means for detecting, classifying, analyzing, categorizing, and assessing calcifying nano-particles. Detecting particular proteins while associated with a calcifying nano-particle can be used to indicate the presence and type of calcifying nano-particle, which can be used to indicate the presence of, or disposition to, diseases or conditions. Multiple proteins on a calcifying particle can be detected. The presence or absence of particular proteins and the pattern of the presence and absence of particular proteins can be used to indicate the presence and type of calcifying nano-particle.

The disclosed method can involve detecting calcifying particles by detecting one or more proteins on the calcifying particle. The method generally can involve detecting at least one protein on the calcifying particle by binding at least one compound to the protein and detecting the bound compound. Binding a compound to the protein can involve, for example, an antibody. The antibody can be the compound and also can be the means of specific binding of the compound to the protein. As another example, a compound can be associated with an antibody with the antibody mediating binding of the compound to the protein. Detecting the bound compound can be accomplished by, for example, detecting the compound directly or indirectly. For example, the compound can be detected using, for example, a microarray, coded beads, flow cytometry, ELISA, mass spectrometry, fluorescence, chemiluminescence, spectrophotometry, chromatography, electrophoresis, or a combination. It should be understood that myriad compositions and methods are known for the detection of analytes and such can be used in and with the disclosed compositions and methods for the detection of calcifying nano-particles and proteins on calcifying nano-particles. Some such compositions and methods are described herein and others are known to those of skill in the art.

It has also been discovered that particular proteins and other components are found on calcifying nano-particles and that detection of such proteins and components can serve to detect, classify, analyze, categorize, and assess calcifying nano-particles. For example, the detection of two or more particular proteins in association (in the same location or on the same particle, for example) is indicative and/or characteristic of calcifying nano-particles. As another example, the detection of a particular protein on a calcifying nano-particle is indicative and/or characteristic of calcifying nano-particles. The presence of the protein on the calcifying nano-particle and/or the identity of combinations of particular proteins serve as identifying characteristics of calcifying nano-particles.

Said proteins can undergo a conformational change as result of being associated with calcifying nano-particles. For example, calcium binding proteins will bind to the mineral calcium or hydroxy apaptite coating that surrounds calcifying nano-particles in the circulatory system of a mammal. There may be primary or primary and secondary changes that occur to the calcium binding protein. Secondary conformational changes involve crosslink formation between peptides or modification of amino acids in peptides by enzymes, oxidation, and chemical reactions. The conformational changes may result in neoepitopes which are specific to these conformationally changed proteins. This specificity of conformational changed proteins on the surface of the calcifying nano-particles provides for the specific discovery, detection, classification, analysis, categorization, and assessment of calcifying nano-particles as described herein is useful for diagnosing, assessing, and/or monitoring diseases associated with calcification and calcifying nano-particles, the progress of such diseases, and the progress of treatment of such diseases.

Calcifying nano-particles are implicated in and represent a risk factor for disease. For example, as described in Example 1, calcifying nano-particles can stimulate a novel blood coagulation mechanism. This mechanism can explain why thrombosis occurs in diseases associated with calcification and calcifying nano-particles. Because of this discovery, detection, classification, analysis, categorization, and assessment of calcifying nano-particles as described herein is useful for diagnosing, assessing, and/or monitoring diseases associated with calcification and calcifying nano-particles, the progress of such diseases, and the progress of treatment of such diseases.

Disclosed is a method for detecting calcifying nano-particles, where the method comprises detecting calcifying nano-particles by detecting one or more proteins on the calcifying nano-particles.

Also disclosed is a method for detecting one or more proteins, where the method comprises detecting one or more proteins on a calcifying nano-particle.

Also disclosed is a method of characterizing a calcifying nano-particle, where the method comprises identifying one or more proteins on a calcifying nano-particle.

Also disclosed is a method of charactering a calcifying nano-particle, where the method comprises identifying one or more protein on a calcifying nano-particle where said identification forms a pattern.

Also disclosed is a method using said pattern to diagnose a disease or condition.

Also disclosed is a method of diagnosing a disease or condition, where the method comprises identifying one or more proteins on a calcifying nano-particle from a subject. The identified proteins identify a disease or condition with which calcifying nano-particles having the identified proteins are related or associated.

Also disclosed is a method of assessing the prognosis of a disease or condition, where the method comprises identifying one or more proteins on a calcifying nano-particle from a subject. The identified proteins identify calcifying nano-particles that are related to or associated with the prognosis of the disease or condition.

Also disclosed is a method of identifying a subject at risk of a disease or condition, where the method comprises identifying one or more proteins on a calcifying nano-particle from a subject. The identified proteins identify calcifying nano-particles that are related to or associated with a risk of developing a disease or condition.

Also disclosed is an isolated calcifying nano-particle, where the calcifying nano-particle comprises one or more of the proteins selected from the group consisting of proteins with a Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF on the calcifying nano-particle. Additionally, proteins that bind to calcium binding proteins may bind to said calcium binding protein/calcifying nano-particles complex including but not limited to Fetuin binding proteins, Thrombin binding proteins, Troponin binding proteins, Tropomyosin binding proteins, GLA Matric binding proteins, Fibrin binding proteins, Kallikrein binding proteins, Factor binding proteins, Matrix metalloprotinease binding proteins, Platelet glycol binding proteins, NF Kappa B binding protein, Factor X binding protein. Table 9 shows representative proteins.

Also disclosed is a composition comprising a calcifying nano-particle and one or more compounds bound to one or more proteins on the calcifying nano-particle.

Also disclosed is a composition of a calcifying nano-particle comprising a hydroxy apatite (mineral calcium phosphate) coating.

Also disclosed is a composition of a calcifying nano-particle comprising said calcifying nanoparticle and a mineral calcium hydroxy apatite coating containing bound proteins that may be conformationally changed.

Also disclosed is a method of determining the progress of treatment of a subject having calcifying nano-particles, where the method comprises detecting one or more proteins on calcifying nano-particles in a sample from the subject, and repeating the detection in another sample from the subject following treatment. A change in the level, amount, concentration, or a combination of calcifying nano-particles in the subject indicates the progress of the treatment of the subject.

Also disclosed are compositions comprising apatite and a coating material, where, for example, the coating material limits exposure of the blood of a subject when the composition is in a subject. The present applications may provide for testing of implants of other devices for the detection of CNPs, for example, stents, prosthetics, artificial valves, etc. Artificial devices are commonly covered with calcific biofilms.

Also disclosed is a method of testing biocompatibility comprising testing blood coagulation in the absence of anticoagulants.

Also disclosed is a method of testing materials that will be exposed to circulating blood for formation of calcific biofilm formation.

For purposes of explanation, the term “protein” is meant to include both proteins in there natural state or proteins that have undergone a conformational change, be it primary or primary and secondary hereafter.

Calcifying nano-particles can be detected by detecting one or more of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. Additionally, proteins that bind to calcium binding proteins may bind to said calcium binding protein/calcifying nano-particles complex including but not limited to Fetuin binding proteins, Thrombin binding proteins, Troponin binding proteins, Tropomyosin binding proteins, GLA Matric binding proteins, Fibrin binding proteins, Kallikrein binding proteins, Factor binding proteins, Matrix metalloprotinease binding proteins, Platelet glycol binding proteins, NF Kappa B binding protein, Factor X binding protein. Table 9 shows representative proteins. Calcifying nano-particles can be detected by detecting two or more proteins on the calcifying nano-particles. Calcifying nano-particles can be detected by detecting one or more proteins with a GLA-containing domain. Calcifying nano-particles can be detected by detecting one or more proteins with a calcium binding domain. Calcifying nano-particles can be captured, identified, or both prior to, simultaneous with, or following detection of one or more of the proteins. Capture or identification of the calcifying nano-particle can indicate that the detected proteins are on the calcifying nano-particles. Calcifying nano-particles can be captured by binding at least one compound to one or more of the proteins, wherein the compound is or becomes immobilized. Calcifying nano-particles can be identified by binding at least one compound to one or more of the proteins, wherein the calcifying nano-particles are separated based on the compound. Calcifying nano-particles can be separated by fluorescence activated sorting.

One or more of the proteins can be detected by binding at least one compound to the protein and detecting the bound compound. Detection of two or more bound compounds can indicate that the proteins to which the compounds are bound are on the calcifying nano-particle. The two or more compounds can be detected in the same location or at the same time. The compounds can be an antibody, where the antibody is specific for the protein. The calcifying nano-particles can comprise calcium phosphate and one or more of the proteins.

The proteins can be detected by detecting any combination of 100 or fewer of the proteins selected from the group consisting of proteins with a Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate. Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. The proteins can be detected by detecting any combination of 75 or fewer of the proteins. The proteins can be detected by detecting any combination of 50 or fewer of the proteins. The proteins can be detected by detecting any combination of 25 or fewer of the proteins. The proteins can be detected by detecting any combination of 10 or fewer of the proteins. The proteins can be detected by detecting any combination of 7 or fewer of the proteins. The proteins can be detected by detecting any combination of 3 or fewer of the proteins. The combination of proteins can be detected in the same assay. The combination of proteins can be detected simultaneously. The combination of proteins can be detected on the same calcifying nano-particle. The combination of proteins can be detected on or within the same device.

The combination of proteins detected can constitute a pattern of proteins. The pattern can indicate or identify a disease or condition, a risk of a disease or condition, the severity of a disease or condition, or a combination. The pattern can indicate or identify a treatment to inhibit, remove or prevent the calcifying nano-particles. The pattern can identify the type of calcifying nano-particles detected.

The proteins can be detected by detecting the presence or absence of any combination of 100 or fewer of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. The pattern of the presence or absence of the proteins can indicate or identify a disease or condition, a risk of a disease or condition, the severity of a disease or condition, or a combination. The pattern of the presence or absence of the proteins can indicate or identify a treatment to inhibit, remove or prevent the calcifying nano-particles. The pattern of the presence or absence of the proteins can identify the type of calcifying nano-particles detected. The presence of one or more of the proteins can indicate or identify a disease or condition, a risk of a disease or condition, the severity of a disease or condition, or a combination. The presence of one or more of the proteins can indicate or identify a treatment to inhibit, remove or prevent the calcifying nano-particles. The presence of one or more of the proteins can identify the type of calcifying nano-particles detected. The absence of one or more of the proteins indicates or identifies a disease or condition, a risk of a disease or condition, the severity of a disease or condition, or a combination. The absence of one or more of the proteins can indicate or identify a treatment to inhibit, remove or prevent the calcifying nano-particles. The absence of one or more of the proteins can identify the type of calcifying nano-particles detected.

The proteins can be detected using any suitable composition, apparatus, or technique, for example, a microarray, coded beads, flow cytometry, ELISA, mass spectrometry, fluorescence, chemiluminescence, spectrophotometry, chromatography, electrophoresis, or a combination.

The proteins on the calcifying nano-particle can be detected by (a) capturing the calcifying nano-particle, (b) binding a detection compound to one or more of the proteins, and (c) detecting the detection compound. The proteins on the calcifying nano-particle can be detected by (a) binding a detection compound to one or more of the proteins, (b) capturing the calcifying nano-particle, and (c) detecting the detection compound. The calcifying nano-particle can be captured by binding a capture compound to one or more of the proteins, where the capture compound is or becomes immobilized. The proteins to which capture compounds bind can mediate capture, where the detection compound can be bound to one of the proteins, where the calcifying nano-particle can be characterized by determining which proteins mediate capture of the calcifying nano-particle to which the detected detection compound is bound. The capture compound can be bound to one of the proteins, where the detection compounds detected can indicate which of the proteins is present on the calcifying nano-particle, where the calcifying nano-particle can be characterized by which proteins are present on the calcifying nano-particle.

The identified proteins can identify the type of calcifying nano-particle. The identified type of calcifying nano-particle can be related to or associated with a disease or condition. The identified proteins can identify a disease or condition with which calcifying nano-particles having the identified proteins are related or associated. The identified proteins can identify a disease or condition that is caused by calcifying nano-particles having the identified proteins. The identified proteins can identify a disease or condition in which calcifying nano-particles having the identified proteins are produced.

Subjects in which pathological thrombosis can occur via apatite-mediated clotting are useful targets for the disclosed methods. Such subjects can include (1) Patients with vulnerable plaque rupture exposing atheroma calcification; (2) Patients undergoing angioplasty or heart-lung machine perfusion; (3) Patients with massive bone fractures or dislocated implants releasing potentially apatite particles; (4) Patients with implants, catheters, wires or stents subject to calcium encrustation; (5) Cancer patients with soft tissue calcification; and (6) Healthy or sick people with CNPs in their blood or positive calcification scores in arteries.

The composition can comprise a calcifying nano-particle and one or more compounds bound to two or more proteins on the calcifying nano-particle. The compound can comprise an antibody, where the antibody is specific for the protein. The compound can block the calcifying nano-particle.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1E are diagrams showing an example of Surface Antigen Pattern Immunoassay (SAPIA). CNP refers to calcifying nano-particles.

FIGS. 2A and 2B are graphs of levels of signal generated for various proteins in SAPIA performed on positive (FIG. 2A) and negative (FIG. 2B) serum and plasma samples showing same levels in serum and plasma.

FIG. 3 is a scatterplot of SAPIA results for clotting matrix GLA proteins, fibrinogen and tissue factor, and CNP capture ELISA results.

FIG. 4 is a graph of levels of signal generated for various proteins in SAPIA showing the presence of pro-thrombin fragments and oesteocalcin in CNPs as measured by sepia.

FIGS. 5A and 5B are graphs of prothrombin activation on apatite using bovine (FIG. 5A) and human (FIG. 5B) prothrombin.

FIG. 6 is a graph of whole blood clotting times for various materials using glass slide test.

FIG. 7 is a diagram of apatite-mediated clotting pathway.

FIG. 8 is a diagram of a model for conformational changes caused by apatite/blood calcium binding as exemplified by prothrombin.

FIG. 9 is a diagram of formation of fibrin in response to thrombotic event due to CNPs how thrombin bound to apatite surface activates formation of fibrin.

FIG. 10A shows boxplots of individual disease states.

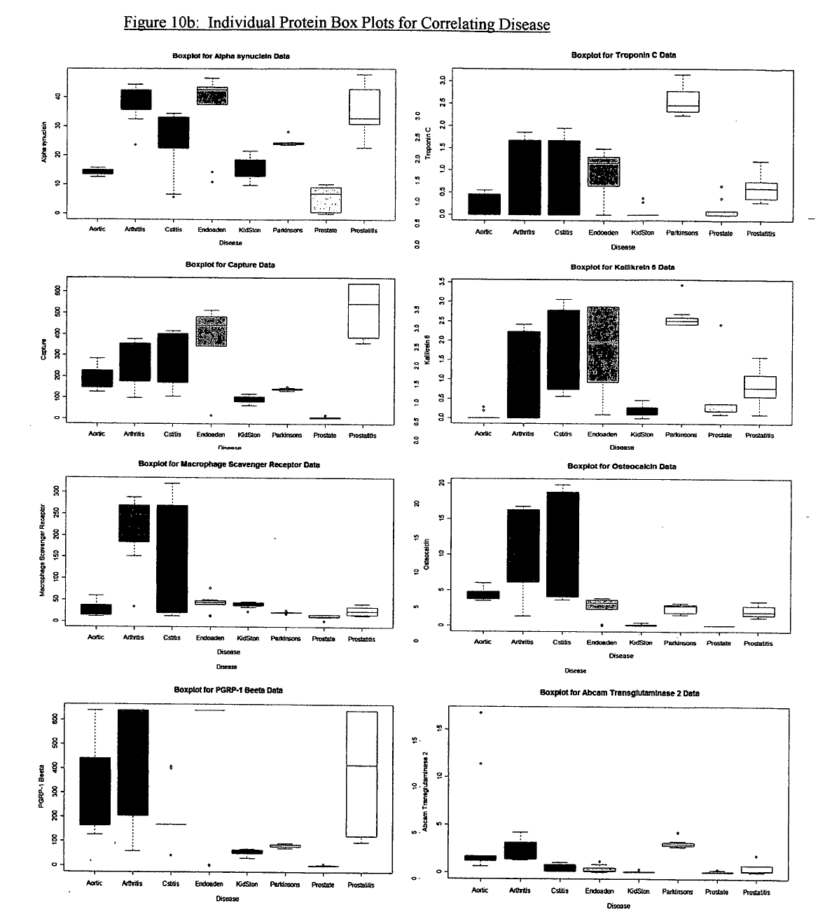

FIG. 10B shows boxplots of individual proteins correlating with disease.

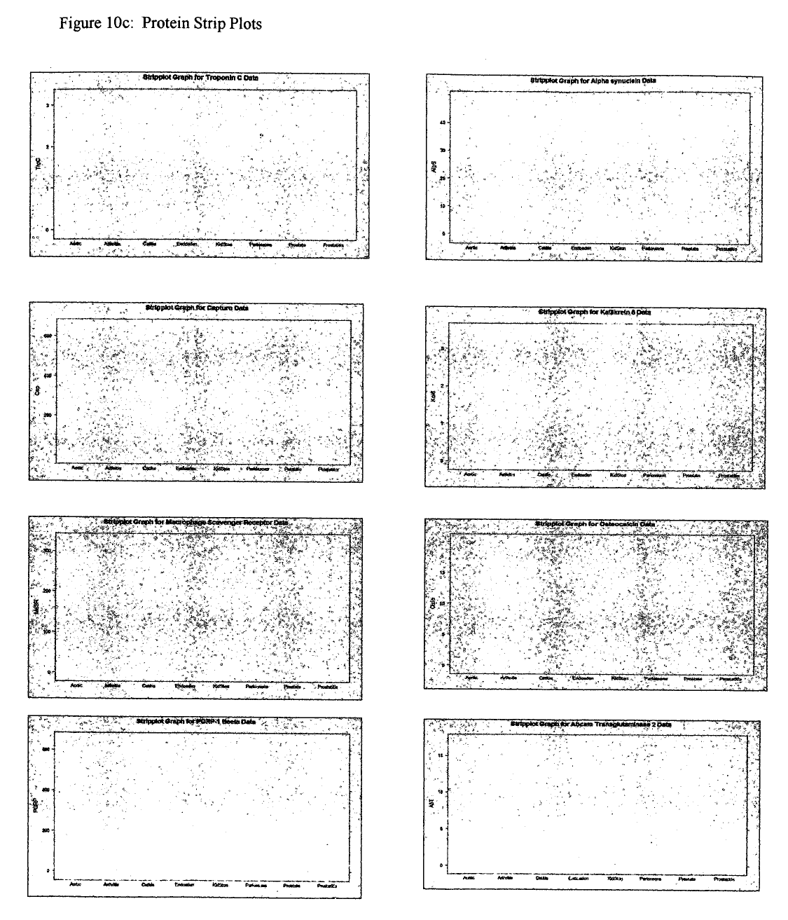

FIG. 10C shows protein stip plots.

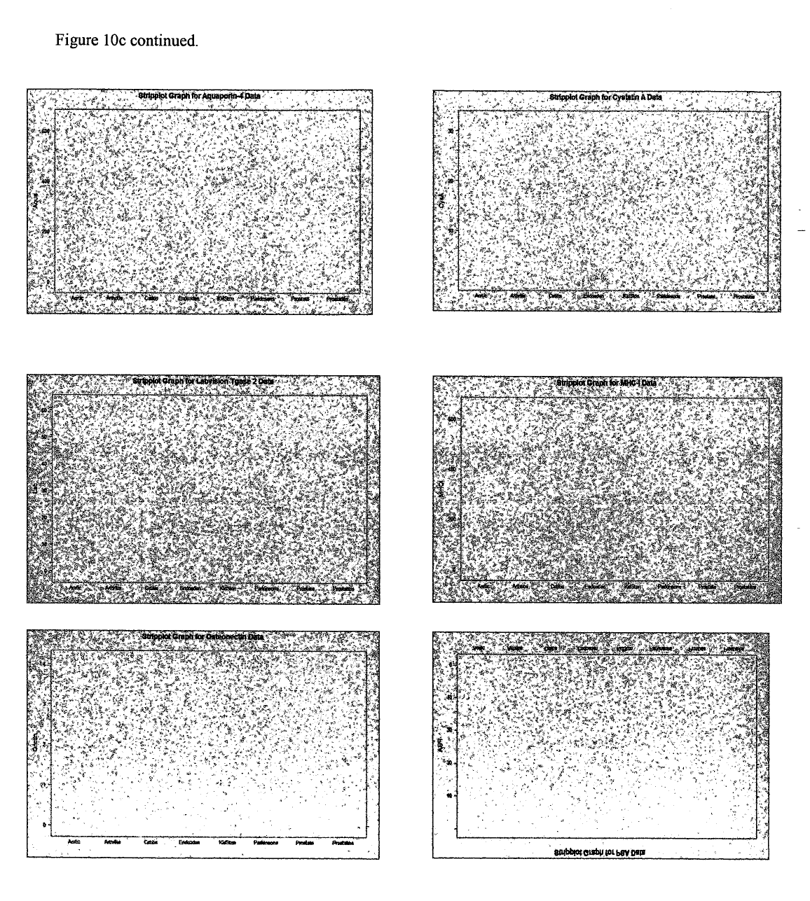

FIG. 11 is a graph of clinomics samples for 15 diseases associated with CNPs. Marker values can be obtained from the disease.

FIG. 12 is a graph depicting urine expression showing physiological differentiations of various CNP isolates ^{99m}Tc.

FIG. 13 is a graph of CNP antigen (U/mL) for Pacreatitis, Rheumatoid Arthritis and Cholecystitis.

FIG. 14 is a boxplot of biomarkers for negative endometrioid adenocarcinoma.

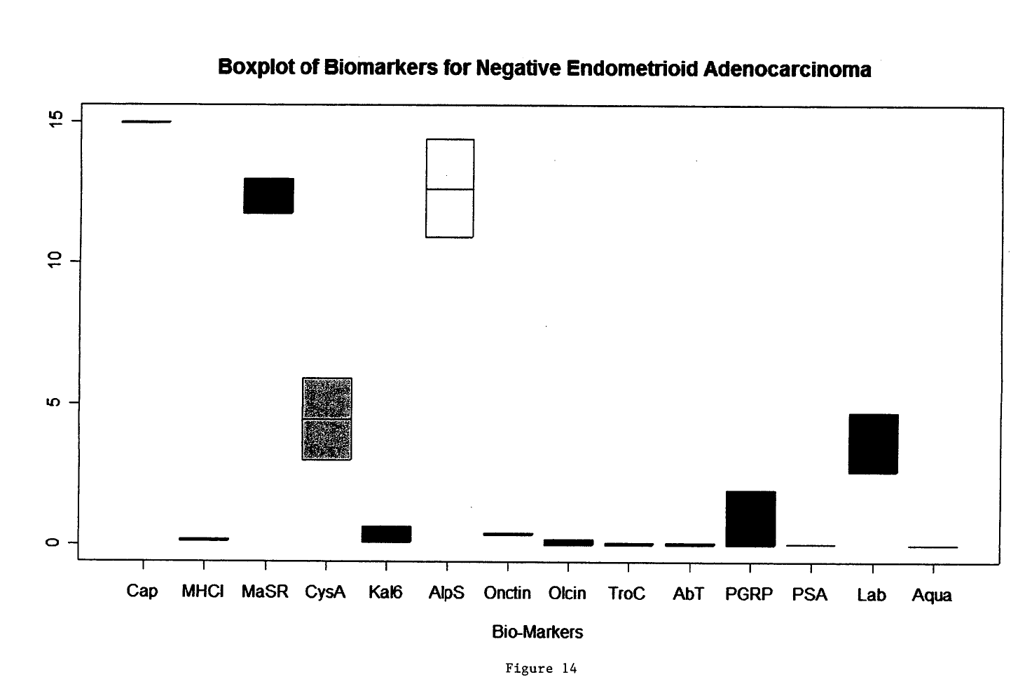

FIG. 15 is a boxplot for biomarkers for positive endometrioid adenocarcinoma.

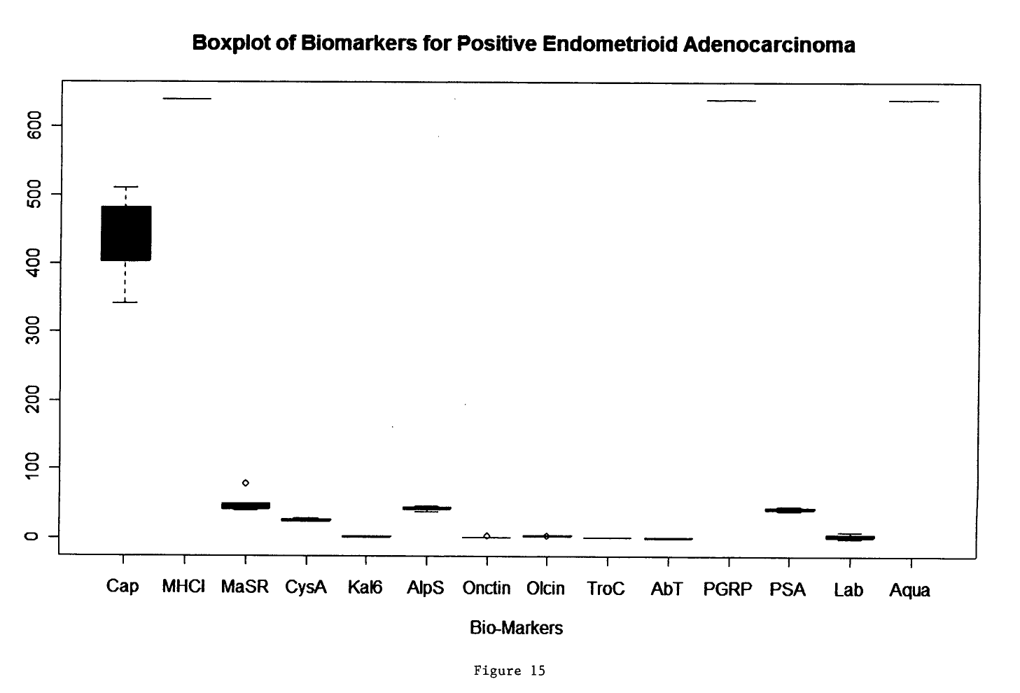

FIG. 16 is scatterplot of markers for aortic data.

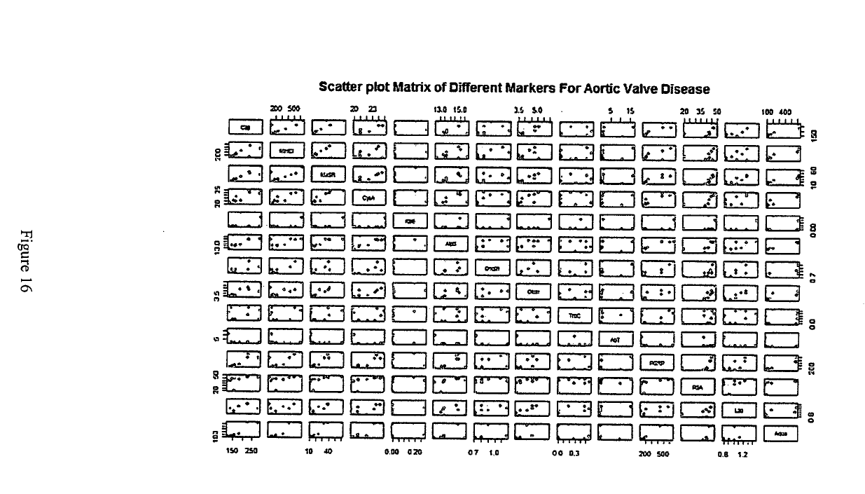

FIG. 17 is a scatterplot of markers for arthritis data.

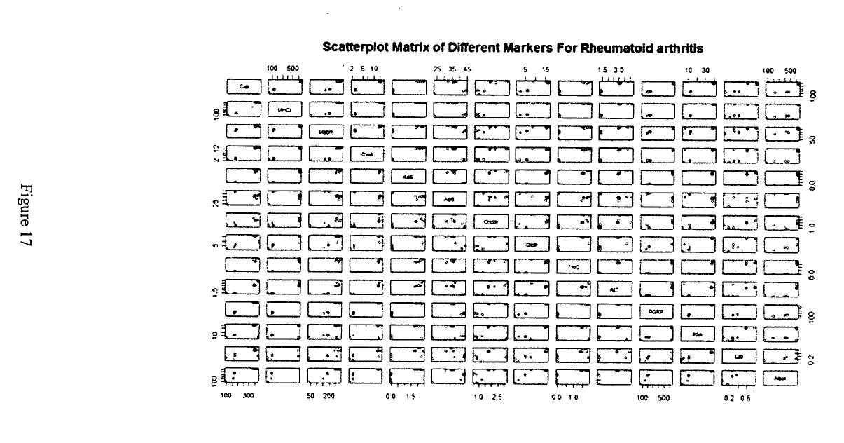

FIG. 18 is a scatterplot of markers for cholecystitis data.

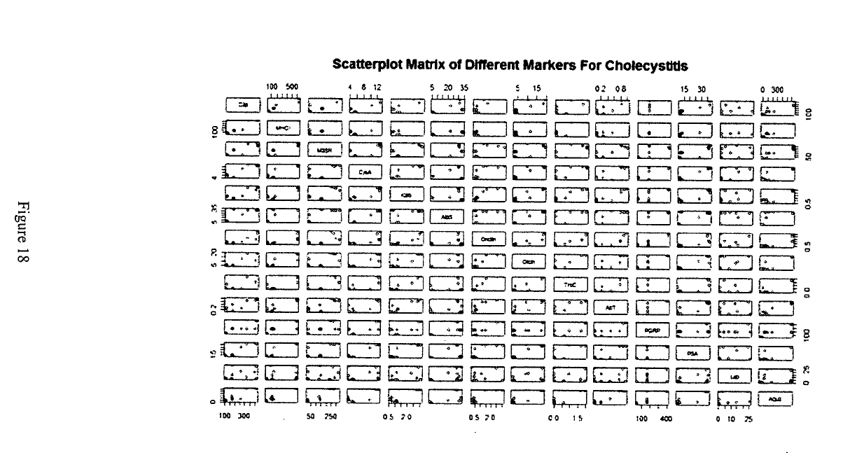

FIG. 19 is a scatterplot of markers for endometrioid data.

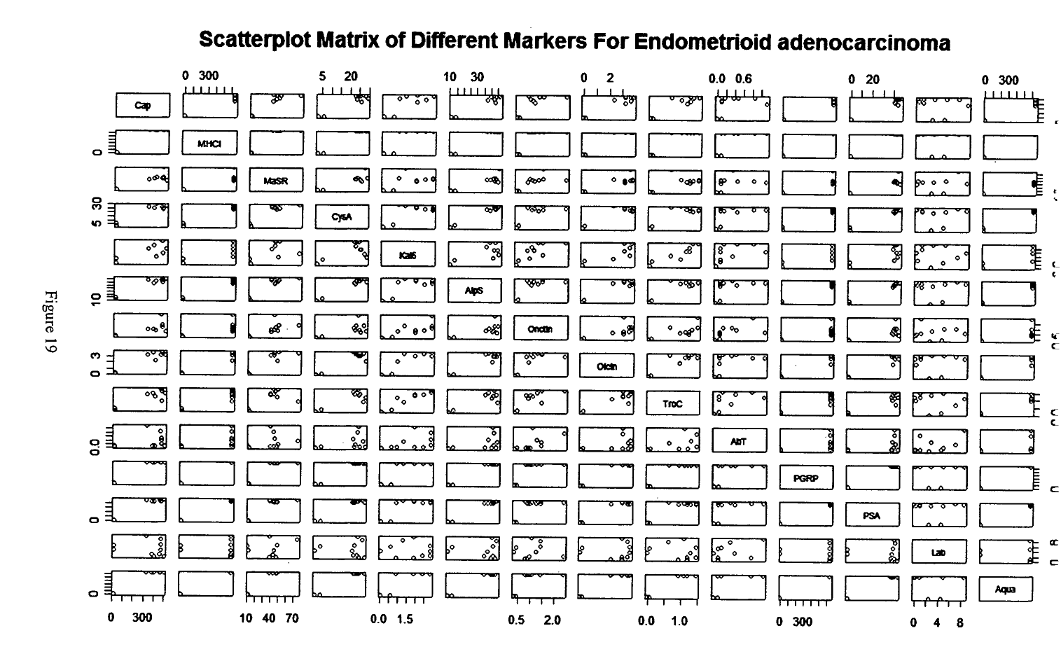

FIG. 20 is a scatterplot of markers for kidney stones data.

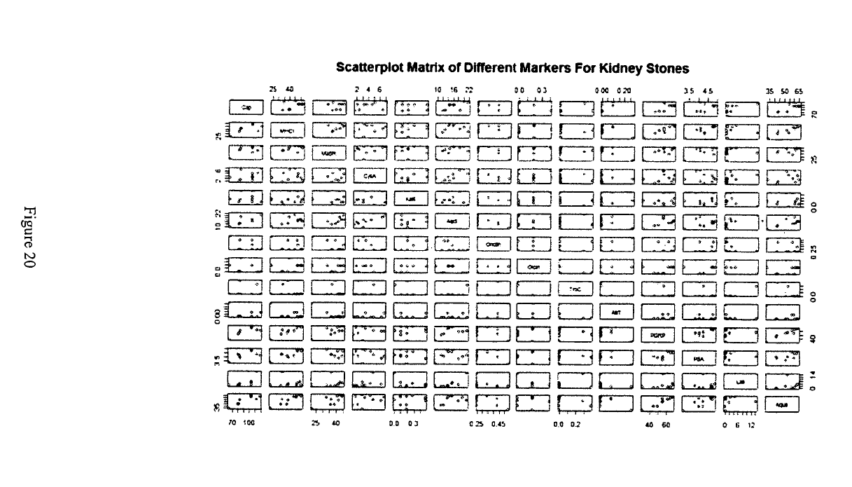

FIG. 21 is a scatterplot of markers for Parkinson's data.

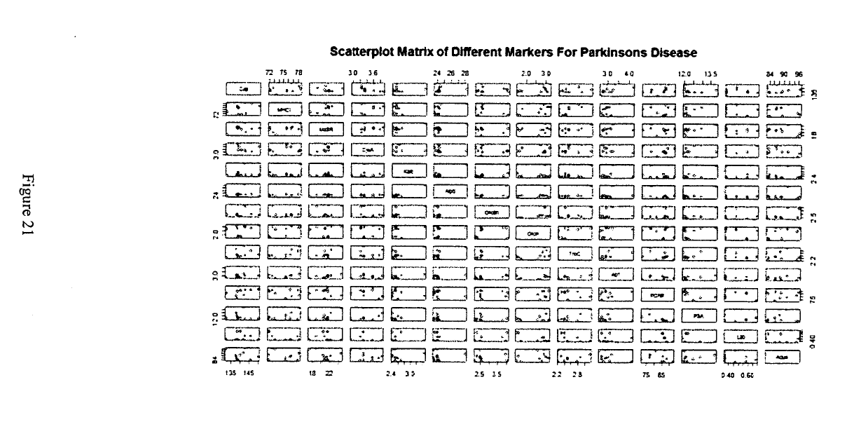

FIG. 22 is a scatterplot of markers for prostate data.

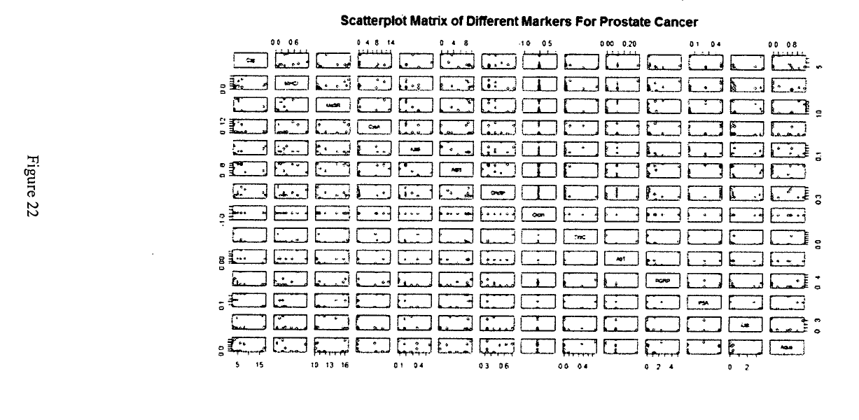

FIG. 23 is a scatterplot of markers for prostatitis data.

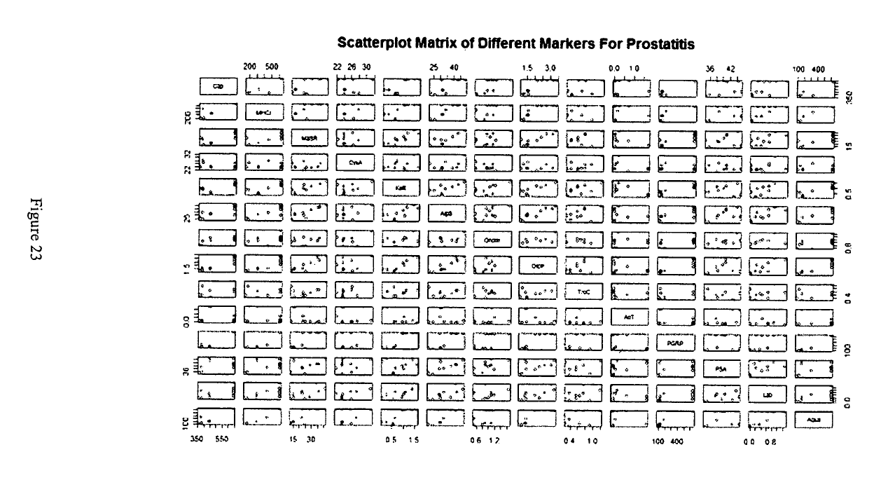

### DETAILED DESCRIPTION OF THE INVENTION

The disclosed method and compositions may be understood more readily by reference to the following detailed description of particular embodiments and the Example included therein and to the Figures and their previous and following description.

Disclosed are methods and compositions for detecting, analyzing and assessing the significance of calcifying nano-particles. The disclosed methods and compositions generally involve detecting one or more proteins present on a calcifying nano-particle. It has been discovered that particular proteins become associated with calcifying nano-particles. This association provides a means for detecting, classifying, analyzing, categorizing, and assessing calcifying nano-particles. Detecting particular proteins while associated with a calcifying nano-particle can be used to indicate the presence and type of calcifying nano-particle, which can be used to indicate the presence of, or disposition to, diseases or conditions. Multiple proteins on a calcifying particle can be detected. Proteins may experience a conformational change resultant from association and/or binding to the calcifying nano-particle. Proteins associated with calcifying nano-particles may undergo secondary conformational changes. Proteins may bind to proteins associated to calcifying nanoparticles. The presence or absence of particular proteins and the pattern of the presence and absence of particular proteins can be used to indicate the presence and type of calcifying nano-particle.

The disclosed method can involve detecting calcifying particles by detecting one or more proteins on the calcifying particle. The method generally can involve detecting at least one protein on the calcifying particle by binding at least one compound to the protein and detecting the bound compound. Binding a compound to the protein can involve, for example, an antibody. The antibody can be the compound and also can be the means of specific binding of the compound to the protein. As another example, a compound can be associated with an antibody with the antibody mediating binding of the compound to the protein. Detecting the bound compound can be accomplished by, for example, detecting the compound directly or indirectly. For example, the compound can be detected using, for example, a microarray, coded beads, flow cytometry, ELISA, mass spectrometry, fluorescence, chemiluminescence, spectrophotometry, chromatography, electrophoresis, or a combination. It should be understood that myriad compositions and methods are known for the detection of analytes and such can be used in and with the disclosed compositions and methods for the detection of calcifying nano-particles and proteins on calcifying nano-particles. Some such compositions and methods are described herein and others are known to those of skill in the art.

It has been discovered that particular proteins and other components are found on calcifying nano-particles and that detection of such proteins and components can serve to detect, classify, analyze, categorize, and assess calcifying nano-particles. For example, the detection of two or more particular proteins in association (in the same location or on the same particle, for example) is indicative and/or characteristic of calcifying nano-particles. As another example, the detection of a particular protein on a calcifying nano-particle is indicative and/or characteristic of calcifying nano-particles. The presence of the protein on the calcifying nano-particle and/or the identity of combinations of particular proteins serve as identifying characteristics of calcifying nano-particles.

Detection of two or more proteins associated with calcifying nanoparticles enables the generation of a patterns that are useful for diagnosing, assessing, and/or monitoring diseases. The origin and activity of said detected proteins is useful in the determination of a potential or active disease state in the host.

Calcifying nano-particles are implicated in and represent a risk factor for disease. For example, as described in the Example, calcifying nano-particles can stimulate a novel blood coagulation mechanism. This mechanism can explain why thrombosis occurs in diseases associated with calcification and calcifying nano-particles. Because of this discovery, detection, classification, analysis, categorization, and assessment of calcifying nano-particles as described herein is useful for diagnosing, assessing, and/or monitoring diseases associated with calcification and calcifying nano-particles, the progress of such diseases, and the progress of treatment of such diseases.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a protein is disclosed and discussed and a number of modifications that can be made to a number of molecules including the protein are discussed, each and every combination and permutation of the protein and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Disclosed is an isolated calcifying nano-particle, where the calcifying nano-particle comprises one or more of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF on the calcifying nano-particle. In addition, binding proteins to the aforementioned protein list can bind to the associated proteins. Proteins may or may not undergo a primary and/or secondary conformational change.

Also disclosed is a composition comprising a calcifying nano-particle where the calcifying nano-particle is covered in a hydroxy apatite (calcium phosphate mineral) coating that collects said calcium binding proteins.

Also disclosed is a composition comprising a calcifying nano-particle where the calcifying nano-particle is covered in a hydroxy apatite (calcium phosphate mineral) coating that has calcium binding proteins associated thereon and proteins that bind to said calcium binding proteins.

Also disclosed is a composition comprising a calcifying nano-particle where the calcifying nano-particle is covered in a hydroxy apatite (calcium phosphate mineral) coating that has calcium binding proteins associated thereon wherein said calcium binding proteins undergo a primary conformation change as a result of said association

Also disclosed is a composition comprising a calcifying nano-particle where the calcifying nano-particle is covered in a hydroxy apatite (calcium phosphate mineral) coating containing bound calcium binding proteins that may experience conformational changes and wherein secondary bound proteins thereon experience conformational changes.

Also disclosed is a composition comprising a calcifying nano-particle and one or more compounds bound to one or more proteins on the calcifying nano-particle.

Also disclosed are compositions comprising apatite and a coating material, where, for example, the coating material limits exposure of the blood of a subject when the composition is in a subject.

The composition can comprise a calcifying nano-particle and one or more compounds bound to two or more proteins on the calcifying nano-particle. The compound can comprise an antibody, where the antibody is specific for the protein. The compound can block the calcifying nano-particle.

A. Compounds

The disclosed method can make use of compounds that can bind to calcifying nano-particles, such as compounds that can bind proteins on calcifying nano-particles. Detection compounds and capture compounds are examples of such compounds. Compounds for use in the disclosed methods can be any compound, molecule, material or substance that can bind to a calcifying nano-particle and/or a protein on a calcifying nano-particle. It is preferred that the compound bind specifically to the calcifying nano-particle or protein. Such specificity allows detection and identification of calcifying nano-particles and proteins. Useful compounds include antibodies and molecules that can bind to proteins on calcifying nano-particles such as ligands, substrates, proteins, cofactors, coenzymes.

Useful compounds include compounds, such as antibodies, that can bind to proteins with a Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF. The disclosed compounds can be used for detection and capture of calcifying nano-particles and/or proteins on calcifying nano-particles. Although not limited to such uses, detecting compounds can be used for detection and capture compounds can be used for capture of calcifying nano-particles and/or proteins on calcifying nano-particles. Detection and identification of calcifying nano-particles and proteins on calcifying nano-particles can be facilitated by including labels on the disclosed compounds. Useful labels and their use are described elsewhere herein. Detection of compounds bound to calcifying nano-particles and/or proteins on calcifying nano-particles indicates the presence of the bound calcifying nano-particles and/or proteins on calcifying nano-particles. The disclosed compounds can be detected, for example, via labels on the compounds, by direct detection of the compounds (via an intrinsic feature of the compounds, for example), or by binding a secondary compound to the primary compound and detecting the secondary compound. For this purpose, the secondary compound can include a label.

B. Labels

To aid in detection, identification, and/or quantitation of calcifying nano-particles and proteins on calcifying nano-particles, labels can be used. For example, labels can be incorporated into, coupled to, or associated with, compounds, detection compound, capture compound (such as compounds to be bound to proteins). A label can include, for example, a fluorescent dye, a member of binding pair, such as biotin/streptavidin, a metal (e.g., gold), or an epitope tag that can specifically interact with a molecule that can be detected, such as by producing a colored substrate or fluorescence. Many other types of labels and signals, and many other principals of signal detection and known and can also be used, some of which are described herein. For example, labels (and other compounds and components) can be detected using nuclear magnetic resonance, electron paramagnetic resonance, surface enhanced raman scattering, surface plasmon resonance, fluorescence, phosphorescence, chemiluminescence, resonance raman, microwave, photometry, mass spectrometry, or a combination.

Substances suitable for detectably labeling proteins include, for example, fluorescent dyes (also known herein as fluorochromes and fluorophores), chromophores, and enzymes that react with colorometric substrates (e.g., horseradish peroxidase). The use of fluorescent dyes is useful as they can be detected at very low amounts. Furthermore, in the case where multiple proteins are to be detected in a single assay, array, and/or system, each protein can be associated with a distinct label compound for simultaneous and/or multiplex detection. Labels can be detected using a detection device or apparatus suitable for the label to be detected, such as a fluorimeter, spectrophotomer, or mass spectrometer, the presence of a signal indicating the presence of the corresponding protein.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G S; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP(S65T); GFP red shifted (rsGFP); GFP wild type non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

Other labels include molecular or metal barcodes, mass labels, and labels detectable by nuclear magnetic resonance, electron paramagnetic resonance, surface enhanced raman scattering, surface plasmon resonance, fluorescence, phosphorescence, chemiluminescence, resonance raman, microwave, photometry, mass spectrometry, or a combination. Mass labels are compounds or moieties that have, or which give the labeled component, a distinctive mass signature in mass spectroscopy. Mass labels are useful when mass spectroscopy is used for detection. Preferred mass labels are peptide nucleic acids and carbohydrates. Combinations of labels can also be useful. For example, color-encoded microbeads having, for example, 256 unique combinations of labels, are useful for distinguishing numerous components. For example, 256 different ligator-detectors can be uniquely labeled and detected allowing multiplexing and automation of the disclosed method.

Examples of useful labels are described in de Haas et al., “Platinum porphyrins as phosphorescent label for time-resolved microscopy,” J. Histochem. Cytochem. 45(9): 1279-92 (1997); Karger and Gesteland, “Digital chemiluminescence imaging of DNA sequencing blots using a charge-coupled device camera,” Nucleic Acids Res. 20(24):6657-65 (1992); Keyes et al., “Overall and internal dynamics of DNA as monitored by five-atom-tethered spin labels,” Biophys. J 72(1):282-90 (1997); Kirschstein et al., “Detection of the DeltaF508 mutation in the CFTR gene by means of time-resolved fluorescence methods,” Bioelectrochem. Bioenerg. 48(2):415-21 (1999); Kricka, “Selected strategies for improving sensitivity and reliability of immunoassays,” Clin. Chem. 40(3):347-57 (1994); Kricka, “Chemiluminescent and bioluminescent techniques,” Clin. Chem. 37(9):1472-81 (1991); Kumke et al., “Temperature and quenching studies of fluorescence polarization detection of DNA hybridization,” Anal. Chem. 69(3):500-6 (1997); McCreery, “Digoxigenin labeling,” Mol. Biotechnol. 7(2):121-4 (1997); Mansfield et al., “Nucleic acid detection using non-radioactive labeling methods,” Mol. Cell. Probes 9(3):145-56 (1995); Nurmi et al., “A new label technology for the detection of specific polymerase chain reaction products in a closed tube,” Nucleic Acids Res. 28(8):28 (2000); Oetting et al. “Multiplexed short tandem repeat polymorphisms of the Weber 8A set of markers using tailed primers and infrared fluorescence detection,” Electrophoresis 19(18):3079-83(1998); Roda et al., “Chemiluminescent imaging of enzyme-labeled probes using an optical microscope-videocamera luminograph,” Anal. Biochem. 257(1):53-62 (1998); Siddiqi et al., “Evaluation of electrochemiluminescence- and bioluminescence-based assays for quantitating specific DNA,” J. Clin. Lab. Anal. 10(6):423-31 (1996); Stevenson et al., “Synchronous luminescence: a new detection technique for multiple fluorescent probes used for DNA sequencing,” Biotechniques 16(6):1104-11 (1994); Vo-Dinh et al., “Surface-enhanced Raman gene probes,” Anal. Chem. 66(20):3379-83 (1994); Volkers et al., “Microwave label detection technique for DNA in situ hybridization,” Eur. J. Morphol. 29(1):59-62 (1991).

Metal barcodes, a form of molecular barcode, can be, for example, 30-300 nm diameter by 400-4000 nm multilayer multi metal rods. These rods can be constructed by electrodeposition into an alumina mold, then the alumina is removed leaving these small multilayer objects behind. The system can have multiple zones encoded using multiple different metals where the metals have different reflectivity and thus appear lighter or darker in an optical microscope depending on the metal. For example, up to 12 zones can be encoded in up to 7 different metals. This allows practically unlimited identification codes. The metal bars can be coated with glass or other material, which can facilitate attachment of the bars to compounds to be labeled. The bars can be identified from the light dark pattern of the barcode.

Epitopes can be used as labels. Epitopes (that is, a portion of a molecule to which an antibody binds) can be composed of sugars, lipids or amino acids. Epitope tags are useful for the labeling and detection of proteins when an antibody to the protein is not available. Due to their small size, they are unlikely to affect the tagged protein's biochemical properties. Epitope tags generally range from 10 to 15 amino acids long and are designed to create a molecular handle for the protein. An epitope tag can be placed anywhere within the protein, but typically they are placed on either the amino or carboxyl terminus to minimize any potential disruption in tertiary structure and thus function of the protein. Any short stretch of amino acids known to bind an antibody could become an epitope tag. Useful epitope tags include c-myc (a 10 amino acid segment of the human protooncogene myc), haemoglutinin (HA) protein, His6, Green flourescent protein (GFP), digoxigenin (DIG), and biotin. Flourescent dyes, such as those described herein, can also be used as epitope tags.

C. Samples

Calcifying nano-particles and proteins on calcifying nano-particles can be any from any source, such as an animal. In general, the disclosed method is performed using a sample that contains (or is suspected of containing) calcifying nano-particles. A sample can be any sample of interest. The source, identity, and preparation of many such samples are known. The sample can be, for example, a sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, and biopsy aspiration. Types of useful samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, and/or crude cell lysate samples.

The sample can be from any organism of interest that contains or is suspected of containing calcifying nano-particles. For example, the sample can be animal, non-human animals, vertebrate, non-human vertebrate, invertebrate, insect, amphibian, avian, reptilian, fish, mammalian, non-human mammalian, rodent, farm animal, domesticated animal, bovine, porcine, murine, feline, canine, or human. The term subject can refer to any animal or any member of any subgroup or classification of animal, including those listed above and elsewhere herein. The term patient can refer to any animal under care or treatment, such as a veterinary patient or human patient.

D. Solid Supports

Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports. A preferred form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A preferred form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of calcifying nano-particles and proteins.

Although preferred, it is not required that a given array be a single unit or structure. The set of compounds may be distributed over any number of solid supports. For example, at one extreme, each compound may be immobilized in a separate reaction tube or container, or on separate beads or microparticles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second, detection compound, such as an antibody. The detection compound can be specific for the same or a different protein on the calcifying nano-particle.

Methods for immobilizing antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy]succinimide ester (GMBS). These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A preferred method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting calcifying nano-particles, the kit comprising one or more detection compounds, one or more capture compounds, and one or more solid supports. The kits also can contain one or more buffers.

F. Mixtures

Disclosed are mixtures formed by performing or preparing to perform the disclosed method. For example, disclosed are mixtures comprising a calcifying nano-particle, a detection compound, and a capture compound.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

G. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a calcifying nano-particle, a detection compound, and a solid support.

H. Data Structures and Computer Control

Disclosed are data structures used in, generated by, or generated from, the disclosed method. Data structures generally are any form of data, information, and/or objects collected, organized, stored, and/or embodied in a composition or medium. A pattern of proteins present on a calcifying nano-particle stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. These include such techniques as neural network that may quickly analyze and interpret data for clinical diagnosis and interpreations to indicated a disease state. Such computer control, computer controlled processes, data structures, and computer programs are contemplated and should be understood to be disclosed herein.

### Uses

The disclosed methods and compositions are applicable to numerous areas including, but not limited to, detecting, analyzing and assessing the significance of calcifying nano-particles. Other uses include, for example, detecting one or more proteins on a calcifying nano-particle, characterizing a calcifying nano-particle, diagnosing a disease or condition, assessing the prognosis of a disease or condition, identifying a subject at risk of a disease or condition, determining the progress of treatment of a subject having calcifying nano-particles, testing biocompatibility comprising testing blood coagulation in the absence of anticoagulants, and testing materials that will be exposed to circulating blood for formation of calcific biofilm formation. Other uses are disclosed, apparent from the disclosure, and/or will be understood by those in the art.

### Method

Disclosed are methods for detecting, analyzing and assessing the significance of calcifying nano-particles. The disclosed methods generally involve detecting one or more proteins present on a calcifying nano-particle. It has been discovered that particular proteins become associated with calcifying nano-particles. This association provides a means for detecting, classifying, analyzing, categorizing, and assessing calcifying nano-particles. Detecting particular proteins while associated with a calcifying nano-particle can be used to indicate the presence and type of calcifying nano-particle, which can be used to indicate the presence of, or disposition to, diseases or conditions. Multiple proteins on a calcifying particle can be detected. The presence or absence of particular proteins and the pattern of the presence and absence of particular proteins can be used to indicate the presence and type of calcifying nano-particle.

The disclosed method can involve detecting calcifying particles by detecting one or more proteins on the calcifying particle. The method generally can involve detecting at least one protein on the calcifying particle by binding at least one compound to the protein and detecting the bound compound. Binding a compound to the protein can involve, for example, an antibody. The antibody can be the compound and also can be the means of specific binding of the compound to the protein. As another example, a compound can be associated with an antibody with the antibody mediating binding of the compound to the protein. Detecting the bound compound can be accomplished by, for example, detecting the compound directly or indirectly. For example, the compound can be detected using, for example, a microarray, coded beads, coated beads, flow cytometry, ELISA, mass spectrometry, fluorescence, chemiluminescence, spectrophotometry, chromatography, electrophoresis, or a combination.

Detection and identification of calcifying nano-particles and proteins on calcifying nano-particles can be facilitated by including labels on the disclosed compounds. Useful labels and their use are described elsewhere herein. Detection of compounds bound to calcifying nano-particles and/or proteins on calcifying nano-particles indicates the presence of the bound calcifying nano-particles and/or proteins on calcifying nano-particles. The disclosed compounds can be detected, for example, via labels on the compounds, by direct detection of the compounds (via an intrinsic feature of the compounds, for example), or by binding a secondary compound to the primary compound and detecting the secondary compound. For this purpose, the secondary compound can include a label.

Disclosed is a method for detecting calcifying nano-particles, where the method comprises detecting calcifying nano-particles by detecting one or more proteins on the calcifying nano-particles.

Also disclosed is a composition comprising a calcifying nano-particle where the calcifying nano-particle is covered in a hydroxy apatite (calcium phosphate mineral) coating that collects said calcium binding proteins.

Also disclosed is a composition comprising a calcifying nano-particle where the calcifying nano-particle is covered in a hydroxy apatite (calcium phosphate mineral) coating that has calcium binding proteins associated thereon and proteins that bind to said calcium binding proteins.

Also disclosed is a composition comprising a calcifying nano-particle where the calcifying nano-particle is covered in a hydroxy apatite (calcium phosphate mineral) coating that has calcium binding proteins associated thereon wherein said calcium binding proteins undergo a primary conformation change as a result of said association

Also disclosed is a composition comprising a calcifying nano-particle where the calcifying nano-particle is covered in a hydroxy apatite (calcium phosphate mineral) coating containing bound calcium binding proteins that may experience conformational changes and s secondary bound proteins thereon that experience conformational changes.

Also disclosed is a method for detecting one or more proteins, where the method comprises detecting one or more proteins on a calcifying nano-particle.

Also disclosed is a method of characterizing a calcifying nano-particle, where the method comprises identifying one or more proteins on a calcifying nano-particle.

Also disclosed is a method of diagnosing a disease or condition, where the method comprises identifying one or more proteins on a calcifying nano-particle from a subject. The identified proteins identify a disease or condition with which calcifying nano-particles having the identified proteins are related or associated.

Also disclosed is a method of assessing the prognosis of a disease or condition, where the method comprises identifying one or more proteins on a calcifying nano-particle from a subject. The identified proteins identify calcifying nano-particles that are related to or associated with the prognosis of the disease or condition.

Also disclosed is a method of identifying a subject at risk of a disease or condition, where the method comprises identifying one or more proteins on a calcifying nano-particle from a subject. The identified proteins identify calcifying nano-particles that are related to or associated with a risk of developing a disease or condition.

Also disclosed is a method of determining the progress of treatment of a subject having calcifying nano-particles, where the method comprises detecting one or more proteins on calcifying nano-particles in a sample from the subject, and repeating the detection in another sample from the subject following treatment. A change in the level, amount, concentration, or a combination of calcifying nano-particles in the subject indicates the progress of the treatment of the subject.

Also disclosed is a method of testing biocompatibility comprising testing blood coagulation in the absence of anticoagulants.

Also disclosed is a method of testing materials that will be exposed to circulating blood for formation of calcific biofilm formation.

Calcifying nano-particles can be detected by detecting one or more of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. Additionally, proteins that bind to the aforementioned protein list may also become associated with the calcifying nanoparticles. Said proteins may or may not undergo conformational changes.

Calcifying nano-particles can be detected by detecting two or more proteins on the calcifying nano-particles. Calcifying nano-particles can be detected by detecting one or more proteins with a GLA-containing domain. Calcifying nano-particles can be detected by detecting one or more proteins with a calcium binding domain. Calcifying nano-particles can be captured, identified, or both prior to, simultaneous with, or following detection of one or more of the proteins. Capture or identification of the calcifying nano-particle can indicate that the detected proteins are on the calcifying nano-particles. Calcifying nano-particles can be captured by binding at least one compound to one or more of the proteins, wherein the compound is or becomes immobilized. Calcifying nano-particles can be identified by binding at least one compound to one or more of the proteins, wherein the calcifying nano-particles are separated based on the compound. Calcifying nano-particles can be separated by fluorescence activated sorting.

One or more of the proteins can be detected by binding at least one compound to the protein and detecting the bound compound. Detection of two or more bound compounds can indicate that the proteins to which the compounds are bound are on the calcifying nano-particle. The two or more compounds can be detected in the same location or at the same time. The compounds can be an antibody, where the antibody is specific for the protein. The calcifying nano-particles can comprise calcium phosphate and one or more of the proteins.

The proteins can be detected by detecting any combination of 10 or fewer of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. Additionally, proteins that bind to the aforementioned protein list may also become associated with the calcifying nanoparticles. Said proteins may or may not undergo conformational changes. The proteins can be detected by detecting any combination of 7 or fewer of the proteins. The proteins can be detected by detecting any combination of 5 or fewer of the proteins. The proteins can be detected by detecting any combination of 3 or fewer of the proteins. The combination of proteins can be detected in the same assay. The combination of proteins can be detected simultaneously. The combination of proteins can be detected on the same calcifying nano-particle. The combination of proteins can be detected on or within the same device.

The combination of proteins detected can constitute a pattern of proteins. The pattern can indicate or identify a disease or condition, a risk of a disease or condition, the severity of a disease or condition, or a combination. The pattern can indicate or identify a treatment to inhibit, remove or prevent the calcifying nano-particles. The pattern can identify the type of calcifying nano-particles detected. Disease associated with patholical clacification include, but are not limited to . . . for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

The proteins can be detected by detecting the presence or absence of any combination of 10 or fewer of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. Additionally, proteins that bind to calcium binding proteins may bind to said calcium binding protein/calcifying nano-particles complex including but not limited to Fetuin binding proteins, Thrombin binding proteins, Troponin binding proteins, Tropomyosin binding proteins, GLA Matric binding proteins, Fibrin binding proteins, Kallikrein binding proteins, Factor binding proteins, Matrix metalloprotinease binding proteins, Platelet glycol binding proteins, NF Kappa B binding protein, Factor X binding protein. Table 9 shows representative proteins. Said proteins may or may not undergo conformational changes.

The pattern of the presence or absence of the proteins can indicate or identify a disease or condition, a risk of a disease or condition, the severity of a disease or condition, or a combination. The pattern of the presence or absence of the proteins can indicate or identify a treatment to inhibit, remove or prevent the calcifying nano-particles. The pattern of the presence or absence of the proteins can identify the type of calcifying nano-particles detected. The presence of one or more of the proteins can indicate or identify a disease or condition, a risk of a disease or condition, the severity of a disease or condition, or a combination. The presence of one or more of the proteins can indicate or identify a treatment to inhibit, remove or prevent the calcifying nano-particles. The presence of one or more of the proteins can identify the type of calcifying nano-particles detected. The absence of one or more of the proteins indicates or identifies a disease or condition, a risk of a disease or condition, the severity of a disease or condition, or a combination. The absence of one or more of the proteins can indicate or identify a treatment to inhibit, remove or prevent the calcifying nano-particles. The absence of one or more of the proteins can identify the type of calcifying nano-particles detected.

Diseases associated with CNPs and pathological calcification include, but are not limited to, for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

The proteins can be detected using any suitable composition, apparatus, or technique, for example, a microarray, coded beads, flow cytometry, ELISA, mass spectrometry, fluorescence, chemiluminescence, spectrophotometry, chromatography, electrophoresis, or a combination.

Specifically, the disclosed method can use an immunassy detecting approximately 100 or fewer different antigens on the same particles using only one tracer antibody for all of said detected target antigens or epitopes. Thereby utilizing only one standard curve to provide quantitation of the target antigens and/or epitopes (focused on the use of the antibody). The disclosed method is especially suitable for biogenic particles, such as CNPs, due to stable surface structure due to crosslinking of proteins and binding to the HA. However, the disclosed method is not limited to CNPs. The disclosed method can be utilized with viruses, spores, bacteria with stable capsules or similar stable substrate, microparticles in blood, plasma, and the like. Noteably, the particles may be captured using antibodies from any source or based on chemical regions from any source.

The proteins on the calcifying nano-particle can be detected by (a) capturing the calcifying nano-particle, (b) binding a detection compound to one or more of the proteins, and (c) detecting the detection compound. The proteins on the calcifying nano-particle can be detected by (a) binding a detection compound to one or more of the proteins, (b) capturing the calcifying nano-particle, and (c) detecting the detection compound. The calcifying nano-particle can be captured by binding a capture compound to one or more of the proteins, where the capture compound is or becomes immobilized. The proteins to which capture compounds bind can mediate capture, where the detection compound can be bound to one of the proteins, where the calcifying nano-particle can be characterized by determining which proteins mediate capture of the calcifying nano-particle to which the detected detection compound is bound. The capture compound can be bound to one of the proteins, where the detection compounds detected can indicate which of the proteins is present on the calcifying nano-particle, where the calcifying nano-particle can be characterized by which proteins are present on the calcifying nano-particle.

The identified proteins can identify the type of calcifying nano-particle. The identified type of calcifying nano-particle can be related to or associated with a disease or condition. The identified proteins can identify a disease or condition with which calcifying nano-particles having the identified proteins are related or associated. The identified proteins can identify a disease or condition that is caused by calcifying nano-particles having the identified proteins. The identified proteins can identify a disease or condition in which calcifying nano-particles having the identified proteins are produced.

Subjects in which pathological thrombosis can occur via apatite-mediated clotting are useful targets for the disclosed methods. Such subjects can include (1) Patients with vulnerable plaque rupture exposing atheroma calcification; (2) Patients undergoing angioplasty or heart-lung machine perfusion; (3) Patients with massive bone fractures or dislocated implants releasing potentially apatite particles; (4) Patients with implants, catheters, wires or stents subject to calcium encrustation; (5) Cancer patients with soft tissue calcification; and (6) Healthy or sick people with CNPs in their blood or positive calcification scores in arteries.

A. Protein Detection and Identification

Some forms of the disclosed methods involve detection and/or identification of calcifying nano-particles and/or proteins on calcifying nano-particles. Molecules of interest—including calcifying nano-particles, proteins, and/or proteins in or on a calcifying nano-particle—can be detected using any suitable technique. Molecules of interest to be detected can be in any sample, any composition or any other context. Detection and identification of calcifying nano-particles and proteins on calcifying nano-particles can be facilitated by including labels on the disclosed compounds. Useful labels and their use are described elsewhere herein. Detection of compounds bound to calcifying nano-particles and/or proteins on calcifying nano-particles indicates the presence of the bound calcifying nano-particles and/or proteins on calcifying nano-particles. The disclosed compounds can be detected, for example, via labels on the compounds, by direct detection of the compounds (via an intrinsic feature of the compounds, for example), or by binding a secondary compound to the primary compound and detecting the secondary compound. For this purpose, the secondary compound can include a label.

Molecules that interact with or bind to the disclosed calcifying nano-particles and proteins, such as antibodies to a protein, can be detected using known techniques. Many suitable techniques—such as techniques generally known for the detection of proteins, peptides and other analytes and antigens—are known, some of which are described herein. These techniques can involve, for example, direct imaging (for example, microscopy), immunoassays, or functional determination. By “functional determination” is meant that a given protein such as a protein that has a function can be detected by the detection of the function. For example, an enzyme can be detected by evaluating its activity on its substrate.

Labeling of proteins and calcifying nano-particles can be either direct or indirect. In direct labeling, the detecting molecule (the compound that binds the protein of interest such as a detecting compound or capture compound) can include a label. Calcifying nano-particles and/or proteins can be contacted with the labeled molecules (such as detection compounds and capture compounds) under conditions effective and for a period of time sufficient to allow the formation of complexes. The complexes can then be generally washed to remove any non-specifically bound labeled molecules, and the remaining label in the complexes can then be detected. Detection of the label indicates the presence of the detecting molecule which in turn indicates the presence of the protein of interest or other analyte. In indirect labeling, an additional molecule or moiety is brought into contact with, or generated at the site of, the complex of the protein of interest and the detecting molecule. For example, a signal-generating molecule or moiety such as an enzyme can be attached to or associated with the detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to the protein of interest can be contacted with the protein complex. The additional molecule can have a label or signal-generating molecule or moiety. The additional molecule can be termed a secondary molecule or compound. If the secondary molecule is an antibody it can be termed a secondary antibody. The complexes can be contacted with the labeled molecules under conditions effective and for a period of time sufficient to allow the formation of secondary complexes. The secondary complexes can then be generally washed to remove any non-specifically bound labeled secondary molecules, and the remaining label in the secondary complexes can then be detected. The additional molecule can also be or include one of a pair of molecules or moieties that can bind to each other, such as the biotin/avidin pair. In this mode, the detecting molecule can include the other member of the pair.

Other modes of indirect labeling include the detection of primary complexes by a two step approach. For example, a molecule (which can be referred to as a first binding agent), such as an antibody, that has binding affinity for the protein of interest can be used to form secondary complexes, as described above. After washing, the secondary complexes can be contacted with another molecule (which can be referred to as a second binding agent) that has binding affinity for the first binding agent, again under conditions effective and for a period of time sufficient to allow the formation of complexes (thus forming tertiary complexes). The second binding agent can be linked to a detectable label or signal-generating molecule or moiety, allowing detection of the tertiary complexes thus formed. This system can provide for signal amplification.

Methods for detecting and measuring signals generated by labels are known. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by measurement or visualization of the product of a reaction catalyzed by the enzyme; antibodies can be detected by detecting a secondary detection label coupled to the antibody. Such methods can be used directly in the disclosed methods. As used herein, detection molecules are molecules which interact with a molecule of interest (such as a calcifying nano-particle and/or proteins) and to which one or more detection labels are coupled. In another form of detection, labels can be distinguished temporally via different fluorescent, phosphorescent, or chemiluminescent emission lifetimes. Multiplexed time-dependent detection is described in Squire et al., J. Microscopy 197(2):136-149 (2000), and WO 00/08443.

Quantitative measurement of the amount or intensity of a label can be used. For example, quantitation can be used to determine if a given label, and thus the labeled component, is present at a threshold level or amount. A threshold level or amount is any desired level or amount of signal and can be chosen to suit the needs of the particular form of the method being performed.

Methods that involve the detection of a substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Assay and detection techniques described herein use various terms, such as antigen, substance, molecule, analyte, etc., to refer molecules of interest that are to be bound or detected. Use of particular terms is not intended to be limiting. Unless the context clearly indicates otherwise, the assays and detection techniques described herein can be used to assay and detect calcifying nano-particles and proteins, such as proteins on calcifying nano-particles and/or proteins on or associated with the proteins on the calcifying nano-particles. As such, the calcifying nano-particles and proteins can be considered the antigen, substance, molecule, analyte, etc. that is bound and/or detected in the assay or detection technique. Assay and detection techniques described herein refer, at various times, to the use of antibodies, such as antibodies that bind to or are specific for antigens, proteins, molecules, etc. Although many forms of the described assays and detection techniques are typically performed using antibodies, the assays and techniques for use in the disclosed methods is not intended to be limiting. Unless the context clearly indicates otherwise, the assays and detection techniques described herein that are described as using (or that commonly used) antibodies can be used with any suitable compound that can bind to the disclosed calcifying nano-particles and proteins.

1. SAPIA

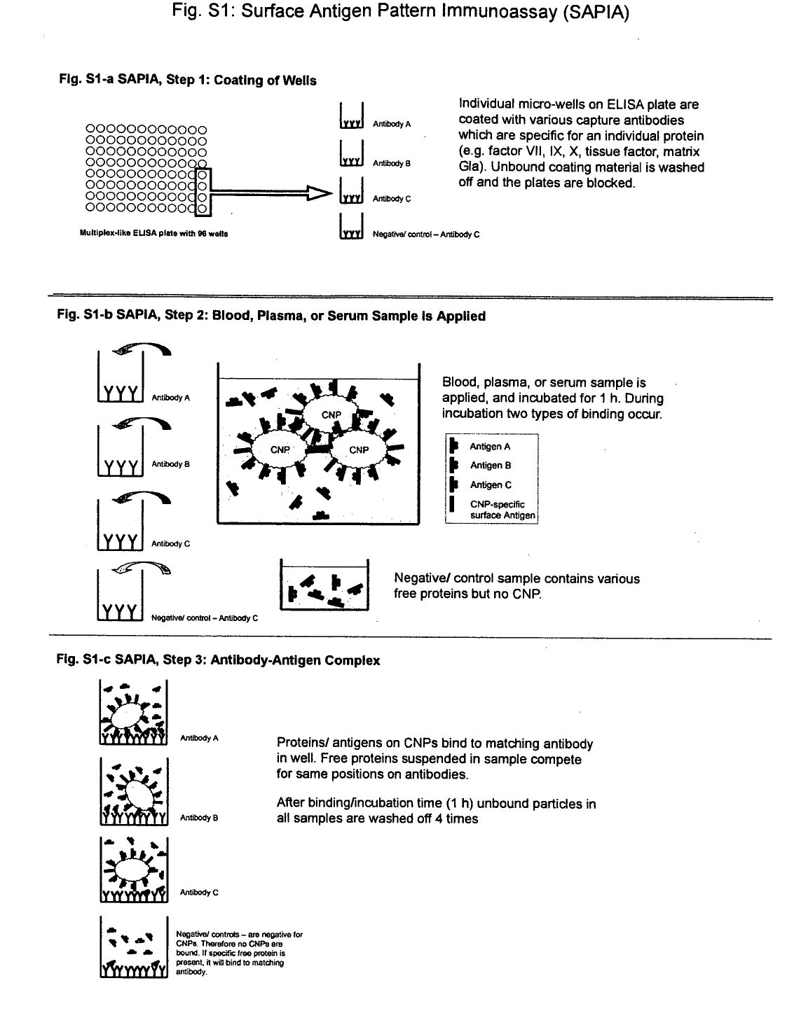

Surface Antigen Pattern Immunoassay (SAPIA) can be used to detect and/or identify stable particles from biological sources and/or components associated thereof. Said components can include, but are not limited to, proteins, peptides, isopeptide bonds, carbohydrates, lipids (fatty acids, phospholipids), endotoxin, heparin sulfate, calcium phosphate, and or nucleic acids (nucleic acid binding proteins associated with HA, amyloid protein P, etc. as associated on the particles.). Examples of stable particles include spores, virus, certain bacteria, any colloidal size mineral, metal biological or synthetic material particles (capable of binding antigens to its surface) and calcifying nanoparticles. For example, SAPIA calcifying nano-particles and/or components on calcifying nano-particles. SAPIA allows detection of the presence of multiple proteins on CNPs (FIGS. 1A-1E). An example and demonstration of SAPIA is described in the Example 1. In SAPIA, capture compounds, such as antibodies specific for one or more proteins on calcifying nano-particles, are immobilized on a solid support. In some forms, capture compounds specific for multiple proteins on calcifying nano-particles can be situated on a single solid support and/or in an array.

SAPIA generally involves capture of calcifying nano-particles on a solid support via binding of one or more proteins on the calcifying nano-particles to capture compounds on the solid support. The captured calcifying nano-particles can then be detected and/or identified by binding a detection compound to the calcifying nano-particles and/or one or more proteins on the calcifying nano-particles and/or one or more of proteins bound to said proteins. In preferred forms of SAPIA, an array of capture compounds specific for different proteins on calcifying nano-particles is used, thus capturing calcifying nano-particles at each array location where a capture compound is present that can bind a protein on the calcifying nano-particles. Because calcifying nano-particles contain a number of proteins, each type of calcifying nano-particle can bind to multiple locations where multiple different capture compounds are present in the array. In this way detection of the presence of calcifying nano-particles at a given array location can identify a protein on the calcifying nano-particle. Such detection can be accomplished with detection compounds that bind to a single type of protein on calcifying nano-particles because only the presence of calcifying nano-particles needs to be detected. In other forms of SAPIA, capture of calcifying nano-particles can be via a single type of protein on calcifying nano-particles and detection can be via multiple types of proteins on calcifying nano-particles or capture and detection can each be via multiple types of proteins on calcifying nano-particles.

2. Immunoassays

Immunodetection methods can be used for detecting, binding, purifying, removing and quantifying various molecules including the disclosed proteins. Further, antibodies and ligands to the disclosed calcifying nano-particles and proteins can be detected. For example, the disclosed proteins can be employed to detect antibodies having reactivity therewith. This is useful, for example, to detect whether a subject has been exposed to or has developed antibodies against a protein. Standard immunological techniques are described, e.g., in Hertzenberg, et al., Weir's Handbook of Experimental Immunology, vols. 1-4 (1996); Coligan, Current Protocols in Immunology (1991); Methods in Enzymology, vols. 70, 73, 74, 84, 92, 93, 108, 116, 121, 132, 150, 162, and 163; and Paul, Fundamental Immunology (3d ed. 1993) each incorporated herein by reference in its entirety and specifically for its teaching regarding immunodetection methods.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986) each incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed proteins. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed calcifying nano-particles and proteins) with an antibody to the molecule of interest or contacting an antibody to a molecule of interest (such as antibodies to the disclosed proteins) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g. antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

The sample used can be any sample that is suspected of containing a molecule of interest (or an antibody to a molecule of interest). The sample can be, for example, one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissue slices, tissue sections, homogenized tissue extract, cell membrane preparation, biopsy aspiration, archeological samples such as bone or mummified tissue, infection samples, nosocomial infection samples, production samples, drug preparation samples, biological molecule production samples, protein preparation samples, lipid preparation samples, and/or carbohydrate preparation samples, and separated or purified forms of any of the above. Such samples can come from subjects or patients.

Immunoassays can include methods for detecting or quantifying the amount of a molecule of interest (such as the disclosed proteins or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each incorporated herein by reference in its entirety and specifically for its teaching regarding immunodetection methods and labels.

Immunoassays that involve the detection of a substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

3. Label-Free Assays

Provided that the concentrations are sufficient, the molecular complexes can be visible to the naked eye, but smaller amounts may also be detected and measured due to their ability to scatter a beam of light. The formation of complexes indicates that both reactants are present, and in immunoprecipitation assays a constant concentration of a reagent antibody can be used to measure specific antigen and reagent antigens can be used to detect specific antibody. If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. A variety of instruments can directly detect molecular interactions (binding, for example). Many are based on an evanescent wave on a sensor surface with immobilized ligand, which allows continuous monitoring of binding.

4. Protein Separation

Detection of calcifying nano-particles and/or proteins can involve the separation of the calcifying nano-particles and/or proteins by electophoresis. In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each herein incorporated by reference in its entirety for its teaching regarding electrophoresis methods.

5. Western Blot

One example of an immunoassay that uses electrophoresis is Western Blot analysis. Western blotting or immunoblotting allows the determination of the molecular mass of a protein and the measurement of relative amounts of the protein present in different samples. Detection methods include chemiluminescence and chromagenic detection. Standard methods for Western Blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each herein incorporated by reference in their entirety for their teaching regarding Western Blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on, the gel. The blot is incubated with a generic protein (such as milk proteins) to bind to any remaining sticky places on the nitrocellulose. An antibody is then added to the solution which is able to bind to its specific protein

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass: proteins are first separated by mass in the SDS-PAGE, then specifically detected in the immunoassay step. Thus, protein standards (ladders) can be run simultaneously in order to approximate molecular mass of the protein of interest in a heterogeneous sample.

6. Capture Assays

Calcifying nano-particles and proteins can be detecting when captured or bound to a solid support (e.g., tube, well, bead, or cell). Examples of such capture assays include Radioimmunoassay (RIA), Enzyme-Linked Immunosorbent Assay (ELISA), Flow cytometry, protein array, multiplexed bead assay, and magnetic capture.

i. Radioimmunoassay (RIA)

Radioimmunoassay (RIA) is a quantitative assay for detection of binding complexes using a radioactively labeled substance (radioligand), either directly or indirectly, to measure the binding of the unlabeled substance to a specific antibody or other compound that can bind to the substance. RIA involves mixing a radioactive substance (because of the ease with which iodine atoms can be introduced into tyrosine residues in a protein, the radioactive isotopes ¹²⁵I or ¹³¹I are often used) with antibody or other compound that can bind to the substance. The antibody or other compound is generally linked to a solid support, such as the tube or beads. Unlabeled or “cold” substance is then adding in known quantities and the amount of labeled substance displaced is measured. Initially, the radioactive substance is bound. When cold substance is added, the two compete for binding sites—and at higher concentrations of cold substance, more binds to the antibody or compound, displacing the radioactive variant. The bound substance is separated from the unbound in solution and the radioactivity of each used to plot a binding curve. The technique is both extremely sensitive, and specific.

ii. ELISAs

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995.; U.S. Pat. No. 4,376,110, each incorporated herein by reference in its entirety and specifically for its teaching regarding ELISA methods. ELISA techniques can also be adapted to use compounds, other than antibodies, that bind to molecules of interest.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface. Such coating and blocking can be used with other capture assays and with other forms of the disclosed methods involving capture and/or solid supports.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control sample to be tested under conditions effective to allow immunecomplex (antigen/antibody) formation. Detection of the immunecomplex then requires a labeled secondary binding agent, or a secondary binding agent in conjunction with a labeled third binding agent.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween. These added agents can also assist in the reduction of nonspecific background.

The “suitable” conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween, or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described elsewhere herein. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

iii. Flow Cytometry

Flow cytometry, fluorescent activated cell sorting (FACS), fluorescence activated sorting, and flow microfluorometry provide a means of scanning individual cells or particles for the presence of a molecule of interest. Although commonly used for analysis of cells, these techniques can be used in the disclosed method to detect, analyze and identify calcifying nano-particles and/or proteins on calcifying nano-particles. Flow Cytometry is the characterization of single cells or particles as they pass at high speed through a laser beam. While a hematologist can count 200 cells in less than a minute by hand (hemocytometer) on a stage microscope, a flow cytometer can discriminate cells at speeds up to 50,000 cells/second. The Flow component is a fluidics system that precisely delivers the cells at the intersection of the laser beam and light gathering lens by hydrodynamic focusing (a single stream of cells is injected and confined within an outer stream at greater pressure). The laser acting as a light source develops parameters of light scatter as well as exciting the fluorescent molecules used to label the cell. Cells are characterized individually by their physical and/or chemical properties (Kohler, G. and Milstein, C. (1975) Continuous Cultures of Fused Cells Secreting Antibody of Predefined Specificity. Nature 256: p. 495-49) which provide analytical parameters capable of accurate quantitation of the number of molecules/cell through Quantitative Flow Cytometry (QFCM). The physical (morphological) profile of a cell or particle can be observed by combining forward light scatter (FS) and orthogonal or side light scatter (SSC). In forward light scatter the laser beam is interrupted by the cell or particle and the light that passes around the cell or particle is measured. Comparable to casting shadow puppets on a wall with a flashlight. This measurement is an indication of the cell's or particle's unique refractive index. Side scatter is the light that is reflected 90° to the laser beam (all fluorescence is emitted and therefore collected at this angle) and is an indication of density or surface granularity.

A short list of some of the information that can be discerned by multiparameter (multi-color) Flow Cytometry includes; Apoptosis (programmed cell death), Cell Type, DNA Content, Enzyme Activity, Intracellular Proteins, Cell Surface Antigens, Cytoplasmic Granularity, Surface Membrane Integrity, Intracellular [Ca++]-Signal Transduction, DNA Synthesis-Proliferation, Cell Surface Receptors, Intracellular Cytokines, Oxidative Metabolism, Intracellular pH, RNA Content, and Cell Size.

Antibodies can provide a useful tool for the analysis and quantitation of markers of individual cells. Such flow cytometric analyses are described in Melamed, et al., Flow Cytometry and Sorting (1990); Shapiro, Practical Flow Cytometry (1988); and Robinson, et al., Handbook of Flow Cytometry Methods (1993), each herein incorporated by reference in its entirety for their teaching regarding FACS. Generally, proteins are detected with antibodies that have been conjugated to fluorescent molecules such as FITC, PE, Texas Red, APC, etc. Molecules on the cell or particle surface can be detected.

By tagging antibodies with a colored fluorochrome, it is easy to distinguish the presence and quantity of antigens on particles or cells. Employing dichroic splitting mirrors, band pass filters and compensation, the colors can be resolved where each color is associated with a single antibody. As each cell or particle, tagged with a fluorescently labeled antibody, enters the laser light outer orbital electrons in the fluorochrome are excited at a specific excitation wavelength (e.g., 494 nm for FITC). As it transitions the width of the laser beam maximum peak fluorescence is achieved within approximately 10 nsec as the excited outer orbital electrons return to their more stable ground state and emit a photon of light at a longer wavelength (e.g., 520 nm for FITC) than that at which they were excited. Photomultiplier tubes (PMT's) detect these faint fluorescent signals and their sole role is to change discrete packets of light called photons (hv) into electrons and amplify them by producing as much as 10 million electrons for every photon captured.

Fluorescence-activated cell sorting (FACS) and fluorescence-activated sorting are types of flow cytometry. FACS is a method for sorting a suspension of biologic cells into two or more containers, one cell at a time. FACS can also be performed on particles such as calcifying nano-particles (in which case it can be referred to as fluorescence-activated sorting). Fluorescence-activated cell sorting is based upon specific light scattering and fluorescence characteristics of each cell or particle. In FACS, the cell or particle suspension is entrained in the center of a narrow, rapidly flowing stream of liquid. The flow is arranged so that there is a large separation between cells and particles relative to their diameter. A vibrating mechanism causes the stream of cells and particles to break into individual droplets. The system is adjusted so that there is a low probability of more than one cell or particle being in a droplet. Just before the stream breaks into droplets the flow passes through a fluorescence measuring station where the fluorescence character of interest of each cell or particle is measured. An electrical charging ring is placed just at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge.

iv. Protein Arrays

Protein arrays are solid-phase ligand binding assay systems using immobilised proteins on surfaces which include glass, membranes, microtiter wells, mass spectrometer plates, and beads or other particles. The assays are highly parallel (multiplexed) and often miniaturised (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems. Such systems and techniques of protein arrays can be used to detect calcifying nano-particles and/or proteins on calcifying nano-particles.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. They may also be used to correlate the polymorphic changes resulting from SNPs with protein function. The capture reagents themselves are selected and screened against many proteins, which can also be done in a multiplex array format against multiple protein targets. Analysis of multiple proteins on calcifying nano-particles can be performed using such techniques.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturisation of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics [Gyros] and specialised chip designs, such as engineered microchannels in a plate [The Living Chip™, Biotrove] and tiny 3D posts on a silicon surface [Zyomyx]. Particles in suspension can also be used as the basis of arrays, providing they are coded for identification; systems include color coding for microbeads [Luminex, Bio-Rad] and semiconductor nanocrystals [QDots™, Quantum Dots], and barcoding for beads [UltraPlex™, Smartbeads] and multimetal microrods [Nanobarcodes™ particles, Nanoplex Technologies]. Beads can also be assembled into planar arrays on semiconductor chips [LEAPS technology, BioArray Solutions].

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system [Prolinx], reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ [PerkinElmer], based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide [Zyomyx] or tantalum pentoxide [Zeptosens].

Array fabrication methods include robotic contact printing, ink-jetting, piezoelectric spotting and photolithography. A number of commercial arrayers are available [e.g. Packard Biosience] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g. antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) [PerkinElmer Lifesciences]. Planar waveguide technology [Zeptosens] enables ultrasensitive fluorescence detection, with the additional advantage of no intervening washing procedures. High sensitivity can also be achieved with suspension beads and particles, using phycoerythrin as label [Luminex] or the properties of semiconductor nanocrystals [Quantum Dot]. A number of novel alternative readouts have been developed, especially in the commercial biotech arena. These include adaptations of surface plasmon resonance [HTS Biosystems, Intrinsic Bioprobes], rolling circle DNA amplification [Molecular Staging], mass spectrometry [Ciphergen, Intrinsic Bioprobes], resonance light scattering [Genicon Sciences] and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially [BD Biosciences Clontech, BioRad, Sigma]. Antibodies for capture arrays are made either by conventional immunisation (polyclonal sera and hybridomas), or as recombinant fragments, usually expressed in E. coli, after selection from phage or ribosome display libraries [Cambridge Antibody Technology, BioInvent, Affitech, Biosite]. In addition to the conventional antibodies, Fab and scFv fragments, single V-domains from camelids or engineered human equivalents [Domantis] may also be useful in arrays.

The term ‘scaffold’ refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A [Affibody], ‘Trinectins’ based on fibronectins [Phylos] and ‘Anticalins’ based on the lipocalin structure [Pieris]. These can be used on capture arrays in a similar fashion to antibodies and may have advantages of robustness and ease of production.

Non-protein capture molecules, notably the single-stranded nucleic acid aptamers which bind protein ligands with high specificity and affinity, are also used in arrays [SomaLogic]. Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labeling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand.

An alternative to an array of capture molecules is one made through ‘molecular imprinting’ technology, in which peptides (e.g. from the C-terminal regions of proteins) are used as templates to generate structurally complementary, sequence-specific cavities in a polymerisable matrix; the cavities can then specifically capture (denatured) proteins which have the appropriate primary amino acid sequence [ProteinPrint™, Aspira Biosystems].

Another methodology which can be used diagnostically and in expression profiling is the ProteinChip® array [Ciphergen], in which solid phase chromatographic surfaces bind proteins with similar characteristics of charge or hydrophobicity from mixtures such as plasma or tumour extracts, and SELDI-TOF mass spectrometry is used to detection the retained proteins. This technology differs from the protein arrays under discussion here since, in general, it does not involve immobilization of individual proteins for detection of specific ligand interactions.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray.

v. Multiplexed Bead Assay

A multiplexed bead assay, such as for example the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

vi. Magnetic Capture

Antibody-coated magnetic particles can be used to capture and selectively separate analytes, such as calcifying nano-particles, from solution. In the technique, target-specific antibody is bound to a magnetic particle (often termed an immunobead). After reaction time to allow binding of immunobead and target, a strong magnetic field is applied to selectively separate the captured target-particle complexes from the milieu.

7. Immunocytochemistry/Immunohistochemistry

Also provided are methods of detecting a substance of interest such as a protein in vivo or in situ using antibody conjugates. Immunocytochemistry and immunohistochemistry are techniques for identifying cellular or tissue constituents, respectively, by means of antigen-antibody interactions. The methods generally involve administering to an animal or subject an imaging-effective amount of a detectably-labeled protein-specific antibody or fragment thereof, and then detecting the location of the labeled antibody in the sample cell or tissue. An “imaging effective amount” is an amount of a detectably-labeled antibody, or fragment thereof, that when administered is sufficient to enable later detection of binding of the antibody or fragment in the specific cell or tissue. The effective amount of the antibody-marker conjugate is allowed sufficient time to come into contact with reactive antigens that are present within the tissues of the subject, and the subject is then exposed to a detection device to identify the detectable marker.

Antibody conjugates or constructs for imaging thus have the ability to provide an image of the tissue, for example, through fluorescence microscopy, laser scanning confocal microscopy (LSCM), magnetic resonance imaging (MRI), SEM, TEM, x-ray imaging, computerized emission tomography and the like.

Fluorescence microscopy and laser scanning confocal microscopy (LSCM) involve the detection of fluorochrome labels, such as those provided herein. Wide-field fluorescence microscopy is a very widely used technique to obtain both topographical and dynamic information. It relies on the simultaneous illumination of the whole sample. The source of light is usually a mercury lamp, giving out pure white light. Optical filters are then used in order to select the wavelength of excitation light (the excitation filter). Excitation light is directed to the sample via a dichroic mirror (i.e., a mirror that reflects some wavelengths but is transparent to others) and fluorescent light detected by a camera (usually a CCD camera). Thus both the illumination and detection of light covering the whole visual field of the chosen microscope objective is achieved simultaneously.

LSCM differs from wide-field fluorescence microscopy in a number of ways. The light source in LSCM is one or more laser(s). This has two consequences. Firstly, the excitation light bandwidth is determined by the source, not the excitation filter and thus is much narrower than in fluorescence microscopy (2-3 nm rather than 20-30 nm). Secondly, in order to illuminate the whole visual field, the laser beam has to be rapidly scanned across the area in a series of lines, much like a TV image is generated. The fluorescence detected at each point is measured in a photomultiplier tube (PMT) and an image built up. The major difference between fluorescence microscopy and LSCM, however, is the pinhole, which is a device that removes unwanted, out-of-focus fluorescence, giving an optical slice of a 3-dimensional image. This “optical slicing” allows the observer to see inside the object of interest and gives clearer images, with more fine detail observable. This method of illumination also has advantages in that it is possible to illuminate selected regions of the visual field allowing complex photobleaching protocols to be carried out to investigate the rates of lateral travel of fluorophores, etc. and for the excitation of different fluorophores in different regions of the same cell. In addition, by altering the focus of the microscope, images can be obtained at different depths. Each image is called a z-section, and can be used to reconstruct an image of the 3-dimensional object.

Multi-Photon LSCM is a variation of LSCM that involves the generation of high energy fluorescence using low energy incident light. This is achieved by delivering multiple photons of excitation light to the same point in space in a sufficiently short time that the energy effectively is summed and so acts as a higher energy single photon. The lasers required for this technique are very specialized and very expensive; however, there are a number of advantages of using multiphoton LSCM over conventional techniques. Firstly, high intensity red light scatters less than low intensity blue light, so objects of interest can be imaged in thicker sections of tissue than in conventional LSCM. Thicker slices are likely to be healthier, and the cells being observed are less likely to have been damaged in the preparation of the sample. Secondly, the lower overall energy of the excitation light means that less phototoxic damage is caused during viewing and less photobleaching is seen, extending the time that cells can be observed. Thirdly, multiphoton LSCM is innately confocal, i.e., no pinhole is required. Excitation of the fluorophore can only occur where the two photons can interact. Given the quadratic nature of the probability of two photons interacting with the fluorophore in the necessary timescale, excitation only occurs in the focal plane of the objective lens, which provides cleaner images.

Elements particularly useful in MRI include the nuclear magnetic spin-resonance isotopes ¹⁵⁷Gd, ⁵⁵Mn, ¹⁶²Dy, ⁵²Cr, and ⁵⁶Fe, with gadolinium often being preferred. Radioactive substances, such as technicium^{99m} or indium¹¹¹, that can be detected using a gamma scintillation camera or detector, also can be used. Further examples of metallic ions suitable for use in the current methods are ¹²³I, ¹³¹I, ⁹⁷Ru, ⁶⁷Cu, ⁶⁷Ga, 125I, ⁶⁸Ga, ⁷²As, ⁸⁹Zr, and ²⁰¹Tl.

A radionuclide can be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA).

Administration of the antibodies can be done as disclosed herein. Nucleic acid approaches for antibody delivery also exist. Antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example. Administration of the antibody can be local or systemic and accomplished intravenously, intra-arterially, via the spinal fluid or the like. Administration also can be intradermal or intracavitary, depending upon the body site under examination. After a sufficient time has lapsed for the labeled antibody or fragment to bind to the diseased tissue, for example 30 minutes to 48 hours, the area of the subject under investigation can then be examined by an imaging technique, such as those described herein.

The distribution of the bound radioactive isotope and its increase or decrease with time can be monitored and recorded. By comparing the results with data obtained from studies of clinically normal individuals, the presence and extent of the diseased tissue can be determined.

The exact imaging protocol will necessarily vary depending upon factors specific to the subject, and depending upon the body site under examination, method of administration, type of label used and the like. One of ordinary skill in the art will be able to determine which imaging protocol to use based on these factors. Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Ferrone et al., Handbook of Monoclonal Antibodies, (1985) ch. 22 and pp. 303-357; Haber et al., Antibodies in Human Diagnosis and Therapy, (1977) pp. 365-389. A typical daily dosage of the antibody used alone might range from about 1 μg/kg to up to 100 mg/kg of body weight or more per day, depending on the factors mentioned above.

As used herein, “FRET” relates to the phenomenon known as “fluorescence resonance energy transfer”. The principle of FRET has been described for example in J. R. Lakowicz, “Principles of Fluorescence Spectroscopy”, 2 (nd) Ed. Plenum Press, New York, 1999. Briefly, FRET can occur if the emission spectrum of a first chromophore (donor chromophore or FRET-donor) overlaps with the absorption spectrum of a second chromophore (acceptor chromophore or FRET-acceptor), so that excitation by lower-wavelength light of the donor chromophore is followed by transfer of the excitation energy to the acceptor chromophore. A prerequisite for this phenomenon is the very close proximity of both chromophores. A result of FRET is the decrease/loss of emission by the donor chromophore while at the same time emission by the acceptor chromophore is observed. A further result of FRET is shortening of the duration of the donor excited state as detected by a reduction in the fluorescence lifetime. A pair of 2 chromophores which can interact in the above described manner is called a “donor-acceptor-pair” for FRET.

In FRET, the energy stored in the excited state of a fluorophore (the donor) upon absorption of a photon, is transferred non-radiatively to a second fluorophore (or chromophore), the acceptor. This transfer is due to dipole-dipole interactions between the emission dipole of the donor and the absorption dipole of the acceptor and depends on the separation distance, the orientation between the dipoles, and the extent of overlapping energy levels (the overlap integral). The inverse sixth order dependence of FRET on separation distance produces an extremely steep decline of the FRET efficiency over a couple of nanometers. Furthermore, the typical distance for most pairs at which 50% of the molecules engage in FRET (the Foerster or R0 distance) lies in the order of magnitude of average protein diameter (4-6 nm), giving rise to detectable FRET at a maximum distance of about 10 mm.

For the latter reason, FRET is a very popular method to assess (fluorescently labeled) protein-protein interactions and protein conformational changes. Thus, FRET can be used to detect calcifying nano-particles and/or proteins on calcifying nano-particles.

A number of techniques are available at present to detect and quantify the occurrence of FRET. These fall into two categories: 1) Spectral, i.e. fluorescence emission intensity-based methods that are based on the loss of donor emission and concomitant gain of acceptor emission. These are: sensitized acceptor emission, ratio imaging, acceptor photobleaching-induced donor unquenching, and anisotropy microscopy. 2) Fluorescence decay kinetics-based methods that are based on the reduced donor photobleaching phenomenon and reduced donor fluorescence lifetime (or duration of the excited state) in the presence of FRET. These are: donor photobleaching kinetics and fluorescence lifetime imaging microscopy (FLIM). The latter technique is especially useful for FRET as the fluorescence lifetime is relatively independent of trivial non-FRET related events and is furthermore independent of fluorophore concentration and light path, both of which are difficult to control in a microscope. Different, functionally equivalent implementations of FLIM exist (Szmacinski et al. (1994) “Fluorescence lifetime imaging microscopy: homodyne technique using high-speed gated image intensifier.” Methods Enzymol, 240:723-48; Wang et al. (1992) Crit Rev Anal Chem, 23 (5): 369-395; Clegg et al. (2003) Methods Enzymol, 360:509-42; Theodorus et al (1993) Biophys. Chem. Volume 48, Issue 2, December 1993, pp. 221-239.)

FRAP (Reits and Neefjes (2001) Nat Cell Biol, June; 3(6):E145-7) is a technique that reports on diffusion of fluorescently labeled biomolecules in living cells. In this technique, a high-power laser beam is used to photodestruct labeled biomolecules in a defined area of the cell. Diffusion (and transport) of molecules from neighboring non-illuminated areas can then repopulate the illuminated area, leading to a time-dependent recovery of fluorescence in this area. For the recovery kinetics, the diffusional recovery can be determined. In another implementation called fluorescence loss in photobleaching (FLIP), the high-power laser illuminates the same area in the cell for a longer period. Diffusionally connected areas in the cell, outside of the illuminated area will loose total fluorescence intensity due to continuous delivery and photodestruction in the illuminated area. FRAP and FLIP can be used to detect and follow the movements of calcifying nano-particles.

A recent implementation of FRAP called fluorescence localization after photobleaching (FLAP) (Dunn et al. (2002) J Microsc, January; 205(Pt 1):109-12) uses two spectrally separated fluorescently labeled forms of the same biomolecule where only one of the fluorescent species is photodestructed, either in the short-term FRAP or long-term FLIP mode. The non-destructed fluorescent species now acts as reference and diffusion/transport of the biomolecule can be assessed by simple division of the two fluorescent emission bands. The loss of the photodestructed species leads to a detectable change in the image ratio and provides, in addition to diffusional velocity parameters, information on the directionality of the diffusion/transport process. The same effect can be achieved when using photoconvertible or photoactivatable fluorescent protein tags (Zhang et al. (2002) Nat Rev Mol Cell Biol, December; 3(12):906-18). This technique is particularly suited to detection of calcifying nano-particles.

### Specific Embodiments

Disclosed is a method for detecting calcifying nano-particles, the method comprising detecting calcifying nano-particles by detecting one or more proteins on the calcifying nano-particles.

Also disclosed is a method for detecting one or more proteins, the method comprising detecting one or more proteins on a calcifying nano-particle.

Also disclosed is a method of characterizing a calcifying nano-particle, the method comprising identifying one or more proteins on a calcifying nano-particle.

Also disclosed is a method of diagnosing a disease or condition, the method comprising identifying one or more proteins on a calcifying nano-particle from a subject, wherein the identified proteins identify a disease or condition with which calcifying nano-particles having the identified proteins are related or associated.

Also disclosed is a method of assessing the prognosis of a disease or condition, the method comprising identifying one or more proteins on a calcifying nano-particle from a subject, wherein the identified proteins identify calcifying nano-particles that are related to or associated with the prognosis of the disease or condition.

Also disclosed is a method of identifying a subject at risk of a disease or condition, the method comprising identifying one or more proteins on a calcifying nano-particle from a subject, wherein the identified proteins identify calcifying nano-particles that are related to or associated with a risk of developing a disease or condition.

Also disclosed is an isolated calcifying nano-particle, wherein the calcifying nano-particle comprises one or more of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C, Macrophage Scavenger Receptor Type I, Antithrombin, Protein S, BAFF on the calcifying nano-particle. In addition, binding proteins to the aforementioned protein list can bind to the associated proteins. Proteins may or may not undergo a primary and/or secondary conformational change.

Also disclosed is a composition comprising a calcifying nano-particle where the calcifying nano-particle is covered in a hydroxy apatite (calcium phosphate mineral) coating that collects said calcium binding proteins.

Also disclosed is a composition comprising a calcifying nano-particle where the calcifying nano-particle is covered in a hydroxy apatite (calcium phosphate mineral) coating that has calcium binding proteins associated thereon and proteins that bind to said calcium binding proteins.

Also disclosed is a composition comprising a calcifying nano-particle where the calcifying nano-particle is covered in a hydroxy apatite (calcium phosphate mineral) coating that has calcium binding proteins associated thereon wherein said calcium binding proteins undergo a primary conformation change as a result of said association

Also disclosed is a composition comprising a calcifying nano-particle where the calcifying nano-particle is covered in a hydroxy apatite (calcium phosphate mineral) coating containing bound calcium binding proteins that may experience conformational changes and wherein secondary bound proteins thereon experience conformational changes.

Also disclosed is a composition comprising a calcifying nano-particle and one or more compounds bound to one or more proteins on the calcifying nano-particle.

Also disclosed is a method of determining the progress of treatment of a subject having calcifying nano-particles, the method comprising detecting one or more proteins on calcifying nano-particles in a sample from the subject, and repeating the detection in another sample from the subject following treatment, wherein a change in the level, amount, concentration, or a combination of calcifying nano-particles in the subject indicates the progress of the treatment of the subject.

Also disclosed herein are compositions comprising apatite and a coating material, where, for example, the coating material limits exposure of the blood of a subject when the composition is in a subject.

Also disclosed herein is a method of testing biocompatibility comprising testing blood coagulation in the absence of anticoagulants and a method of testing materials that will be exposed to circulating blood for formation of calcific biofilm formation.

The calcifying nano-particles can be detected by detecting one or more of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF on the calcifying nano-particles.

The calcifying nano-particles can be detected by detecting two or more proteins on the calcifying nano-particles. The calcifying nano-particles can be detected by detecting one or more proteins with a GLA-containing domain. The calcifying nano-particles can be detected by detecting one or more proteins with a calcium binding domain. The calcifying nano-particles can be captured, identified, or both prior to, simultaneous with, or following detection of one or more of the proteins. Capture or identification of the calcifying nano-particle can indicate that the detected proteins are on the calcifying nano-particles. The calcifying nano-particles can be captured by binding at least one compound to one or more of the proteins, wherein the compound is or becomes immobilized. The calcifying nano-particles can be identified by binding at least one compound to one or more of the proteins, wherein the calcifying nano-particles are separated based on the compound. The calcifying nano-particles can be separated by fluorescence activated sorting.

One or more of the proteins can be detected by binding at least one compound to the protein and detecting the bound compound. Detection of two or more bound compounds can indicate that the proteins to which the compounds are bound are on the calcifying nano-particle. The two or more compounds can be detected in the same location or at the same time. At least one of the compounds can be an antibody, wherein the antibody is specific for the protein. The calcifying nano-particles can comprise calcium phosphate and one or more of the proteins.

The proteins can be detected by detecting any combination of 10 or fewer of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer, Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF on the calcifying nano-particle. The proteins can be detected by detecting any combination of 100 or fewer of the proteins. The proteins can be detected by detecting any combination of 5 or fewer of the proteins. The proteins can be detected by detecting any combination of 3 or fewer of the proteins. The combination of proteins can be detected in the same assay. The combination of proteins can be detected simultaneously. The combination of proteins can be detected on the same calcifying nano-particle. The combination of proteins can be detected on or within the same device.

The combination of proteins detected can constitute a pattern of proteins. The pattern can indicate or identify a disease or condition, a risk of a disease or condition, the severity of a disease or condition, or a combination including but not limited to for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease. The pattern can indicate or identify a treatment to inhibit, remove or prevent the calcifying nano-particles. The pattern can identify the type of calcifying nano-particles detected.

The proteins can be detected by detecting the presence or absence of any combination of 10 or fewer of the proteins selected from the group consisting of proteins Bovine CaBP-HA complex, Fetuin A, Calmodulin, Tissue Transglutaminase II, MMP-9, MMP-3, CD 42b, NF-kappa B, Osteopontin, Factor X/Xa, CD14, Prothrombin, Factor IX, Fetuin B, CD40, Myeloperoxidase, Fibronectin, Factor VII, Tissue factor, Human complement 5b-9, Human CRP, Matrix GLA protein, CD61, Kappa Light Chain, Macrophage L1 Protein, Factor XIIIA, hsp 60, Fibrillin-1, B2 microglobulin, CD 18, Laminin, Antitrypsin, Notch-1, BSA, LBP, PTX3, Complement C5, Fibrinogen, D-Dimer Factor V, gamma-G1a residues, TF-VIIa, Complement 3c3, Complement C4, Antichymotrypsin, Annexin V, Lipid A, Isopeptide bond, Vitronectin, Thrombin, Osteocalcin, Troponin T, Vimentin, Tropomyosin, HAS, Troponin I cardiac, Apo A1, MHC class I, Amyloid P protein, sCD40 L, Kallikrein 6, Prothrombin F1, Antithrombin III, Thrombin, Factor VIII, Heparan Sulphate, Factor XI, c-jun, Fra-2, Fra-1, Jun B, P-c-Jun, TGase3, alpha fetoprotein, Prostate Specific Antigen, erbB2, VEGF, alpha synuclein, Mucin-1, Cystatin A, Cystatin S, Prostein, Aquaporin 4, Trypsin, Tgase 2, Ubiquitin, TLR 4, Cathepsin D, GFAP, RAGE, CD 9, Prostate Acid Phosphatase, Smith Antigen, PRGP-1 Beeta, PGRP-S, Gram positive bacteria, Troponin C Cardiac, Protein C Macrophage Scavenger Receptor Type I, Antithrombin, Protein S, BAFF on the calcifying nano-particle. The pattern of the presence or absence of the proteins can indicate or identify a disease or condition, a risk of a disease or condition, the severity of a disease or condition, or a combination. The pattern of the presence or absence of the proteins can indicate or identify a treatment to inhibit, remove or prevent the calcifying nano-particles. The pattern of the presence or absence of the proteins can identify the type of calcifying nano-particles detected. The presence of one or more of the proteins can indicate or identify a disease or condition, a risk of a disease or condition, the severity of a disease or condition, or a combination. The presence of one or more of the proteins can indicate or identify a treatment to inhibit, remove or prevent the calcifying nano-particles. The presence of one or more of the proteins can identify the type of calcifying nano-particles detected. The absence of one or more of the proteins indicates or identifies a disease or condition, a risk of a disease or condition, the severity of a disease or condition, or a combination. The absence of one or more of the proteins can indicate or identify a treatment to inhibit, remove or prevent the calcifying nano-particles. The absence of one or more of the proteins can identify the type of calcifying nano-particles detected.

The proteins can be detected using a microarray, coded beads, coated beads, flow cytometry, ELISA, mass spectrometry, fluorescence, chemiluminescence, spectrophotometry, chromatography, electrophoresis, or a combination.

The proteins on the calcifying nano-particle can be detected by (a) capturing the calcifying nano-particle, (b) binding a detection compound to one or more of the proteins, and (c) detecting the detection compound. The proteins on the calcifying nano-particle can be detected by (a) binding a detection compound to one or more of the proteins, (b) capturing the calcifying nano-particle, and (c) detecting the detection compound. The calcifying nano-particle can be captured by binding a capture compound to one or more of the proteins, where the capture compound is or becomes immobilized. The proteins to which capture compounds bind can mediate capture, where the detection compound can be bound to one of the proteins, where the calcifying nano-particle can be characterized by determining which proteins mediate capture of the calcifying nano-particle to which the detected detection compound is bound. The capture compound can be bound to one of the proteins, where the detection compounds detected can indicate which of the proteins is present on the calcifying nano-particle, where the calcifying nano-particle can be characterized by which proteins are present on the calcifying nano-particle.

The identified proteins can identify the type of calcifying nano-particle. The identified type of calcifying nano-particle can be related to or associated with a disease or condition. The identified proteins can identify a disease or condition with which calcifying nano-particles having the identified proteins are related or associated. The identified proteins can identify a disease or condition that is caused by calcifying nano-particles having the identified proteins. The identified proteins can identify a disease or condition in which calcifying nano-particles having the identified proteins are produced.

Subjects in which pathological thrombosis can occur via apatite-mediated clotting are useful targets for the disclosed methods. Such subjects can include (1) Patients with vulnerable plaque rupture exposing atheroma calcification; (2) Patients undergoing angioplasty or heart-lung machine perfusion; (3) Patients with massive bone fractures or dislocated implants releasing potentially apatite particles; (4) Patients with implants, catheters, wires or stents subject to calcium encrustation; (5) Cancer patients with soft tissue calcification; and (6) Healthy or sick people with CNPs in their blood or positive calcification scores in arteries. Such people in the last category can be identified using the disclosed compositions and methods.

The composition can comprise a calcifying nano-particle and one or more compounds bound to two or more proteins on the calcifying nano-particle. The compound can comprise an antibody, wherein the antibody is specific for the protein. The compound can block the calcifying nano-particle.

### EXAMPLES

### A. Example 1

In this example evidence is presented of host molecules involving two families of calcium binding G1a-proteins, calcification-defense system and clotting G1a-proteins, simultaneously binding to apatite surfaces and calcifying nano-particles. Thus, it was discovered that both G1a-systems participate in the body's calcification-defense by spatially blocking apatite surfaces. It was also realized that this creates a novel clotting mechanism. Thrombosis (the clotting of blood within an artery or vein) is a major cause of death and serious illness. Patients with circulatory, autoimmune and renal diseases, diabetes and cancers have abnormal ongoing coagulation often leading to thrombosis. However the principle clotting pathways, intrinsic and extrinsic, do not fully explain how or why thrombosis blocks blood vessels. It has been discovered that exposed calcium phosphate mineral surfaces (such as on calcifying nano-particles) simultaneously bind clotting and mineral-defense proteins creating a clotting mechanism that combines both intrinsic and extrinsic factors to the common factor X (FX) and prothrombin activation. This is the first direct proof of a link between calcification, which is widespread in disease, and pathological clotting. It was discovered that this novel physiological clotting mechanism can be activated when apatite is injected intravenously in vivo. A clotting test was developed to measure effects of various surfaces, including apatite and calcifying nano-particles (CNPs), on blood clotting in vitro. A multiplex surface antigen pattern test was also developed to demonstrate the pattern of clotting factors and their activators on the surface of CNPs isolated from human plasma and serum. This multiplex surface antigen pattern test is an example of the disclosed method for detecting calcifying nano-particles. The significance of this novel calcium mediated clotting mechanism is far-reaching since many diseases have a thrombotic component which may cause death.

Clinical experience in cardiovascular medicine suggested that contact of blood with exposed calcified surface leads to thrombi (Halloran and Bekavac, Neuroimaging. 2004 October; 14(4):385-7; Demer, Int. J. Epidemiol. 31, 737 (2002); Bini et al., Arterioscler. Tromb. Vasc. Biol. 19, 1852 (1999); Lahey and Horton, Am J Kidney Dis. 40, 416 (2002)). Recent coronary artery calcification (CAC) scoring data supports that view, because positive CAC scores are a biomarker to predict future atherosclerotic thrombotic events, such as myocardial infarcts and strokes (Demer, Int. J. Epidemiol. 31, 737 (2002)). Many studies have also shown that patients with calcification-associated diseases such as atherosclerosis, kidney and autoimmune diseases, diabetes and cancer often have abnormal ongoing blood coagulation and thrombosis (Doherty et al., Endocrine Reviews 25, 629 (2004); Caine et al., Neoplasia 4, 465 (2002); Chambers and Laurent, Biochem. Soc. Transact. 30, 194 (2002)). Yet, prior to the present discoveries, there was an absence of experimental studies that link calcification directly to thrombus formation.

Current theories on blood clotting involve the binding of tissue factor to factor VIIa to provide for an enzymatically active complex which then activates factors IX and X, leading to thrombin generation and clot formation (Banner et al., Nature 380, 41 (1996)). Injury that exposes tissue factor under the endothelium is the key activator resulting in production of factor Xa. Vitamin-K-dependent, gamma-carboxyglutamic acid (G1a)-containing domains of clotting proteins in this family are homologous and are responsible for phospholipid membrane association considered to be the substratum for clotting activation cascades (Nelsestuen, Trends Cardiovasc. Med. 9, 162 (1999)). Normal hemostasis results in platelet activation, aggregation and more thrombin generation (Dumas et al., Science 301, 222 (2003)) leading to a clot covering the damaged area. Clot growth is stopped by anticoagulation cascades activating inhibitors of clotting. Furthermore, factor Xa and thrombin are assumed to diffuse through the developing clot, filled with their specific inhibitors, to the surface of the growing clot. Formation of a large thrombus blocking a blood vessel is difficult to explain with this hypothesis, and has been experimentally shown to be insufficient (Hathcock and Nemerson, Blood 104, 123 (2004)). Therefore, other explanations have been sought, e.g. the existence of a small pool of circulating tissue factor, factor X, or thrombin, as microvesicles, has been proposed (Del Conde et al., Blood 106, 1604 (2005)).

Four clotting factors (Factor II, Factor VII, Factor IX, Factor X) acting as proteolytic executors of the clotting cascade are calcium-binding proteins also known to bind to apatite/calcium phosphate via their calcium binding G1a domains. The classical models imply that the G1a-domains undergo calcium dependent conformation changes before or concomitant with binding to phospholipid membrane. It was discovered that calcium phosphate surfaces serve the dual function as a suitable substratum (replacing phospholipid membrane) and as activators in normal and pathological blood clotting. The significance of this for human (patho)physiology is high because there are many situations where calcium phosphate minerals can have contact with blood/clotting factors: (1) Acutely during bone fracture, bone surgery, and dental surgery; (2) Artificially with the introduction of uncoated implants, fillers and apatite adjuvants; (3) Chronically with the growth of calcium phosphate deposits in atherosclerotic vessels, catastrophically with rupture of vulnerable plaque, and via cell death, exposing pathological calcification (e.g. Randall's plaque) and stones; (4) Hematologically with calcium phosphate macromolecular complexes with matrix G1a-protein and fetuin that were detected by Price in blood of animals suffering massive bone degradation (Price et al., J. Biol. Chem. 278, 22153 (2003))—Price (Price et al., J. Biol. Chem. 279, 1594 (2004)) showed that blood calcium phosphate macromolecular complexes lead to rapid arterial calcification in vivo; and (5) Systemically with calcifying nano-particles (CNPs), known until now as nanobacteria. CNPs have been found circulating in blood and implicated in pathological calcification (Kajander and Ciftcioglu, Proc. Natl. Acad. Sci. USA. 95, 8274 (1998)). Although CNPs are controversial in their content and genetic characterization, critics and proponents alike agree that CNPs have a calcium phosphate mineral surface (Kajander and Ciftcioglu, Proc. Natl. Acad. Sci. USA. 95, 8274 (1998); Cisar et al., Proc. Natl. Acad. Sci. USA. 97, 11511 (2000); Vali et al, Geochim. Cosmochim. Acta 65, 63 (2001); Miller et al., Am. J. Physiol. Heart Circ. Physiol. 287, H115 (2004); Ciftcioglu et al., Kidney Int. 67, 483 (2005)).

1. Materials and Methods

i. Preparation of Apatite and CNPs

Hydroxyapatite was prepared according to Poser and Price (Poser and Price, J. Biol. Chem. 254, 43 (1979)) using sterile solutions. Calcifying nano-particles (CNPs) were isolated from fetal bovine serum (FBS) obtained from several manufacturers (Seralab, UK; Gibco, Paisley, Scotland; HyClone, Logan, Utah and Biological Industries, Israel) using methods described earlier (Kajander and [symbol] iftçioglu, Proc. Natl. Acad. Sci. USA 95, 8274 (1998)).

ii. Acute Toxicity

Colloidal solutions of synthetic apatite (Poser and Price, J. Biol. Chem. 254, 43 (1979)) or CNPs in sterile phosphate buffered saline (PBS; 0.14M NaCl, 2.7 mM KCl, 8 mM Na₂HPO₄, 1.5 mM KH₂HPO₄, pH 7.4) were injected (at a dose of 0-50 μl wet pellet) into tail vein of Wistar rats. Rats were euthanized with phenobarbital 48 hours later and tissue samples were excised and placed to a fixative, 4% paraformaldehyde, within 2 minutes from start of anesthesia. Tissues were processed to paraffin blocks, sectioned, deparaffinized and stained with H&E and with TUNEL assay for apoptotic changes with In situ Cell Death Detection Kit, AP (Roche) according to the manufacturer's instructions. Tissues were pretreated for TUNEL staining with 20 μg/ml proteinase K (Sigma, molecular biology grade) in 10 mM Tris/HCl, pH 7.4 for 15 min at room temperature. Apoptotic changes were evaluated with light microscopy. The method used detected apoptosis based on labeling of DNA strand-breaks using modified nucleotide labeling by terminal deoxynucleotidyl transferase visualized with enzymic reaction using Fast Red substrate (Roche). No changes were observed in control rats exposed to sterile PBS. The study was approved by the Ethics Committee of the University of Kuopio.

iii. Thrombosis Detection After i.v. Injection of ^{99m}Tc-Labeled Apatite or CNPs in Rabbits

Biodistribution study data on ^{99m}Tc-radiolabeled CNPs and apatite by Akerman et al. (Akerman et al., Proc. SPIE Int. Soc. Opt. Eng. 3111, 436 (1997)) was re-evaluated to detect thrombotic changes after i.v. injection. Dynamic imaging data (1 frame/minute) revealed thrombosis associated with radiolabeled material. Thrombus formation was observed in pulmonary artery on first pass through pulmonary circulation, and was Verified at autopsy at 48 hours.

iv. Whole Blood Clotting Slide Test

Clotting induced by apatite was detected initially by using standard whole blood clotting time tube tests, with added glass beads, incubated at +37° C. water bath with or without apatite. The clotting times were of the order of 2 minutes and did not allow precise evaluation of subtle changes by extraneous materials on clotting time due to need of sample preparation time, such as mixing the extraneous minerals. This could not be amended by using anti-coagulated blood samples (citrate or EDTA), reconstituted with 25 to 50 mM CaCl₂ at start of the test, because such samples clotted poorly indicating irreversible interference by the anticoagulant to some important player(s) in clotting.

A novel test platform was developed using glass slides (Menzel-Glaser, Braunschweig, Germany) incubated at room temperature. The method allows for measuring changes in the clotting time induced by contact with foreign surfaces, i.e. plain glass or coated glass, and for studying the effects of drugs on the clotting induced by foreign surface. Glass slides were coated with synthetic apatite (Poser and Price, J. Biol. Chem. 254, 43 (1979)) and controlled by TEM and EDX analysis (Kajander and ( [symbol] iftçioglu, Proc. Natl. Acad. Sci. USA 95, 8274 (1998)). 250 μl of apatite colloidal suspension (10% pellet containing suspension) was pipetted to each slide, and slides were dried +37° C. overnight. Commercial heat-fixed CNP-coated slides were obtained from Nanobac Oy, Kuopio, Finland. Plain glass slides without further processing were used as a foreign surface. Effect of Calcium EDTA, disodium EDTA, and clodronate on blood clotting time was investigated by adding 10 μl solution to a plain glass slide immediately before addition of blood. Calcium EDTA and disodium EDTA were from Fluka. Clodronate was a gift from Professor Jouko Vepsäläinen (University of Kuopio).

Venous blood was collected with venipuncture from 19 random volunteers participating in CNP epidemiological study (Ethical Committee Approval, Kuopio University). Volunteers signed an informed consent. Blood was collected with venipuncture in siliconized glass serum tubes, EDTA plasma tubes or citrate plasma tubes (Terumo), and was tested immediately after collection for whole blood clotting time on different test platforms.

200 μl of freshly drawn blood was added to pre-coated or plain glass slides which were put on a tilting shaker 15 tilts/minute at room temperature until formation of a solid clot was observed as “frozen droplet”. Experiments were carried out usually in triplicate to determine reproducibility of tests. CV % for the triplicate tests were less than 6.81% (CNP-coated slide, n=13), 8.79% (apatite-coated slide, n=15) and 10.37% (non-treated control slide, n=15). Clotting time results were analyzed by Bonferroni (Dunn) t Test (Table 1).

v. Proteomics on Proteins Bound to Apatite Particles

Protein-free apatite particles in DMEM (Gibco) without any additives were suspended into 10% FBS-DMEM and were immediately centrifuged at 14 000 rpm, 30 rain at +4° C. The pellet was washed two times by suspending with sterile PBS followed by centrifugation at 13,200 rpm, 20 min at room temperature. Pellet was frozen prior to analysis. Proteomics analysis was provided by Protana, Montreal, Canada. The SDS-boiled samples were subjected to 1D SDS-PAGE under reducing conditions. Protein bands were detected by Coomassie staining, excised and processed following standard procedures including:
1. The proteins in the gel plug were reduced with DTT.
2. The free cysteine residues were alkylated with iodoacetamide.
3. The proteins were digested with the endoprotease trypsin.
4. The peptides produced were extracted in neutral, acidic and basic conditions.

vi. Mass Spectrometry Analysis

The peptide mixtures were separated by C18 reverse phase chromatography into a Thermo-Finnigan LTQ-FT ion trap/FTICR hybrid mass spectrometer coupled with a nano-spray interface. The mass spectrometer was operated in data-dependent mode to obtain tandem (ms/ms) spectra of each peptide above an intensity threshold as it emerged from the chromatography column. The raw data files were processed using LCQ-DTA to generate peak lists of the tandem spectra. The processed data was searched with Mascot (Matrix Sciences, London UK) using the NCBI non-redundant database. The Mascot results were curated by mass spectrometry scientists to correlate the results with the raw data (Table 2).

vii. Nanocapture and SAPIA ELISA Methods

Presence of CNPs was first detected with commercially available Nanocapture ELISA kit (Nanobac Oy). The test measures presence of CNPs in human serum or plasma, with a measurement range from 0 to 640 units (Pretorius et al., HIV Med. 5, 391 (2004)). The capture kit uses separate step-wise capture and detection reactions involving two monoclonal antibodies targeted on different surface epitopes on the CNPs.

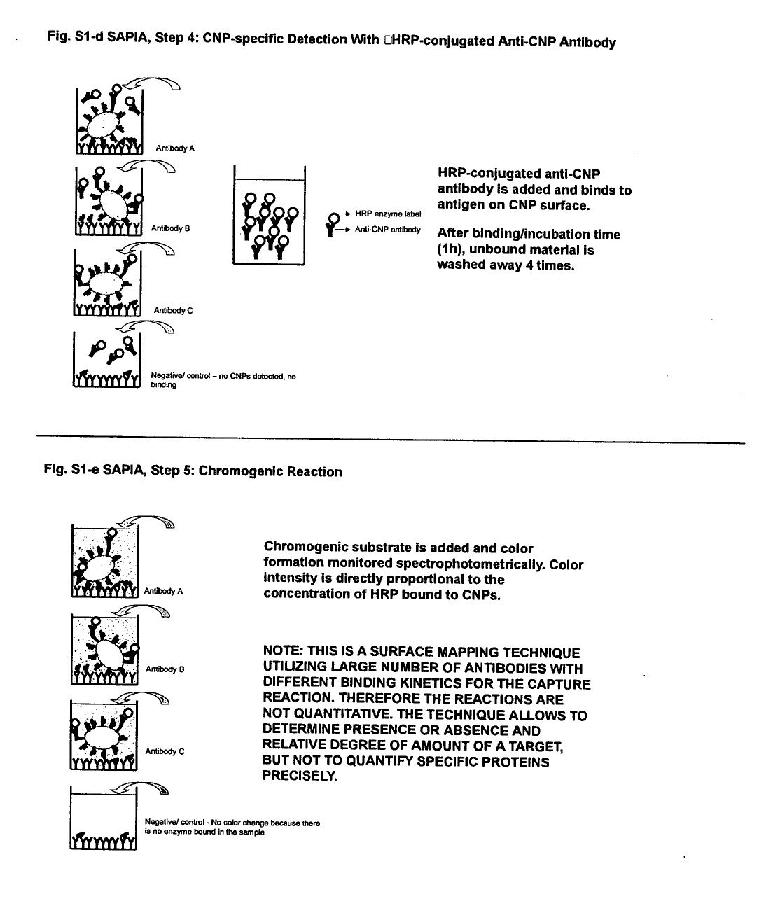

Surface Antigen Pattern Immunoassay (SAPIA) test was developed to detect presence of multiple proteins on CNPs (FIGS. 1A-1E). The SAPIA test here used specific commercial antibodies against designated targets (Table 3), in this case against clotting and anti-calcification G1a-proteins (FIG. 1A). SAPIA method was first tested using plasma and serum samples from 8 persons. Human plasma and serum samples were first tested to determine if they are comparable samples (FIG. 2). Note that the results of positive sample shown are exceptionally high for clotting factors (FIG. 2A). Based on the results obtained with these 8 samples, both serum and plasma samples could be used to test the specified parameters. Thereafter, a random panel (n=16, each sample was combined from 1-5 human serum samples of similar CNP unit values to make up the necessary volume needed to run many tests, done in duplicate) with Nanocapture results ranging from 0-640 Units was selected for the SAPIA test.

SAPIA plates were made by coating high binding polystyrene ELISA plates (Corning, USA) with antibodies against anti-calcification proteins and G1a clotting factors and control antibodies. SAPIA was controlled by using antibodies against human serum albumin, D-Dimer, NF-κB and fibronectin as these proteins were not expected to be specifically bound on particle surface (FIG. 1A). Monoclonal antibodies were diluted at a final concentration of 1 μg/ml with 1×PBS, pH 7.4, 100 μl/well to ELISA plates and incubated at +4° C. overnight. Polyclonal antibodies were diluted to a concentration of 10 μg/ml and plates were coated as above. After coating procedure, plates were washed once with TBS-Tween 20 and blocked by adding TBS-Tween 20 300 μl/well and incubated 2 hours at room temperature. Thereafter, blocking solution was removed and storage solution 0.05% NaN₃-TBS was added 200 μl/well, and the plates were stored sealed with tape in a refrigerator.

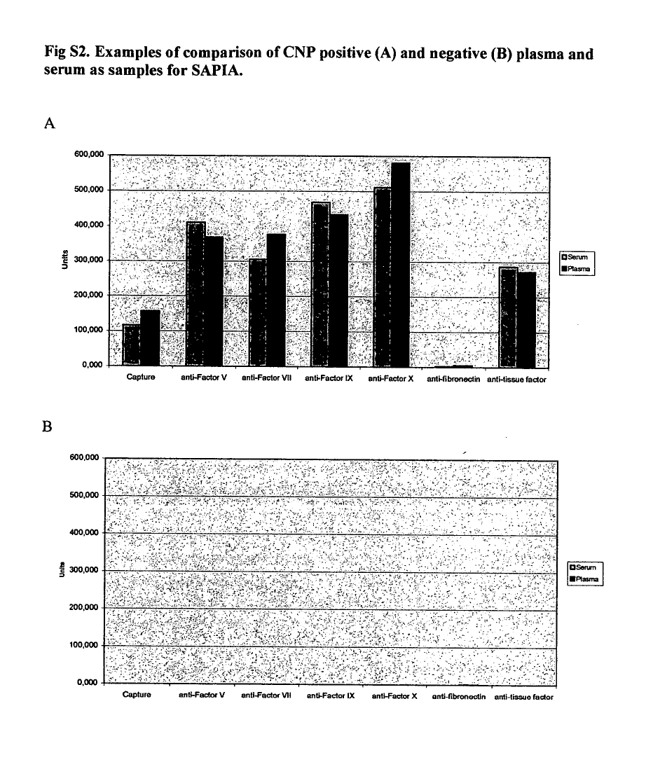

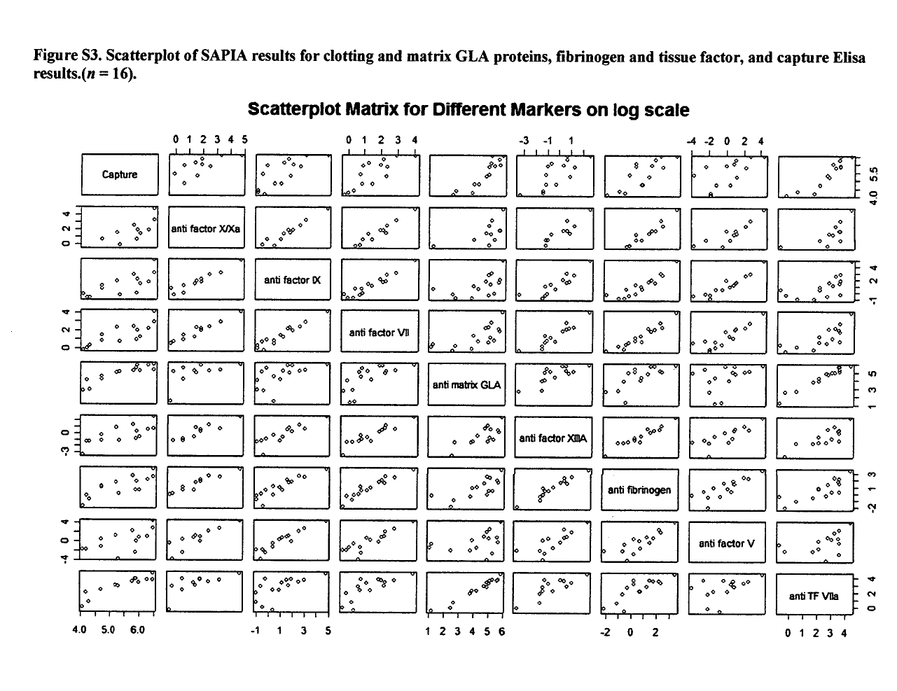

Before use, storage solution was removed and plates were washed once with TBS-Tween. 50 μl/well of Assay Buffer (0.05 M Tris, 0.15 M NaCl, 0.05% Proclin 300, pH 7.5 with 1% mouse serum) was added in duplicates and 50 μl/well serum sample was added (FIG. 1B). Plate was sealed with tape and incubated 1 hour at room temperature with moderate shaking (FIG. 1C). Plates were washed four times using TBS-Tween and detection antibody HRP-8D10 (Nanobac Oy) was added 100 μl/well. Plates were incubated 1 hour at room temperature with moderate shaking (FIG. 1D). Plates were washed four times using TBS-Tween and TMB substrate (Moss Inc., Pasadena, Md. 21123) was added 100 μl/well. Plates were sealed and protected from light with foil and incubated 20 min at room temperature with moderate shaking (FIG. 1E). Absorbance at 630 nm was read with microplate reader (Biohit BP 808). Blank values were subtracted and unit values were calculated from standard curve of Nanocapture ELISA test using TableCurve 2D program (Systat, Point Richmond, Calif.). Pearson Correlation Coefficients (N=16, Prob>|r| under H0: Rho=0) were calculated (Tables 4 and 5). Anti-calcification proteins and coagulation G1a factors were found to be present in CNPs (FIGS. 3 and 4).

viii. Activation of Prothrombin by Apatite In Vitro

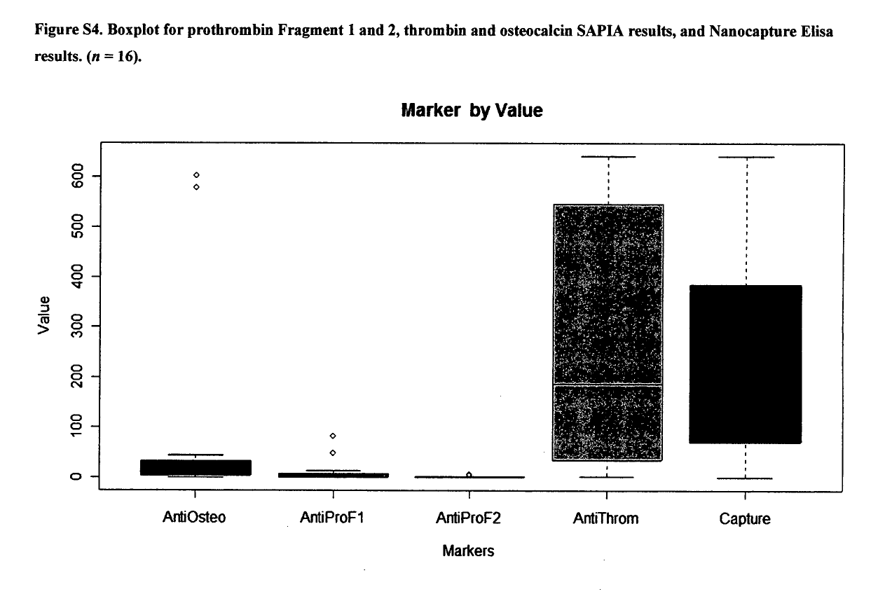

Human prothrombin >95% pure (Calbiochem) and two samples of bovine prothrombin >98% pure (ICN, Aurora, Ohio and American Diagnostica, Stamford, Conn.) were diluted to a concentration of 10 μg/ml, 1 μg/ml and 0.1 μg/ml in 25 mM Tris, 150 mM NaCl and 5 mM CaCl₂, pH 7.4 (which is the substrate buffer for thrombin). 20 μl of prothrombin solution was mixed with 20 μl apatite (Poser and Price, J. Biol. Chem. 254, 43 (1979)) and incubated 30 minutes with moderate shaking at room temperature. As control reactions, prothrombins at above mentioned concentrations were incubated with buffer only. Thereafter, thrombin substrate Sar-Pro-Arg-pNA (Bachem, Bubendorf, Switzerland) 0.5 mg/ml (in substrate buffer solution, first solubilized in acetone) was added (100 μl) to all wells and plate was transferred to +37° C. Reaction was monitored with Elisa reader at 405 nm. Control absorbances were subtracted to produce data shown in FIG. 5. No thrombin generation was found to take place in prothrombin wells without hydroxyapatite addition.

ix. Thrombin and FXa Activity Measurements from SAPIA Tests

Serum and plasma samples from 6 healthy volunteers were used for measurement of thrombin and FXa activity in particles captured with SAPIA using plates coated with antibodies against CNPs, thrombin and Factor X/Xa. 50 μl of serum or plasma samples were pipetted onto plates and 50 μl of Assay Buffer (0.05 M Tris, 0.15 M NaCl, 0.05% Proclin 300, pH 7.5 with 1% mouse serum) was added. Plates were incubated 1 hour at room temperature with moderate shaking. Plates were washed 4 times, before 100 μl specific substrate was added. Three substrates were used for thrombin: Bx-Phe-Val-Arg-pNA HCl (Bachem), Sar-Pro-Arg-pNA (Bachem) and β3-Ala-Gly-Arg-pNA-acetate (Sigma, St. Louis, Mo.). One substrate was used for Factor Xa, CH₃-D-CHA-Gly-Arg-pNA-AcOH (Sigma). Thrombin substrates Bx-Phe-Val-Arg-pNA HCl (0.136 mg/ml) and Sar-Pro-Arg-pNA (0.25 mg/ml) were in 25 mM Tris, 150 mM NaCl, 5 mM CaCl₂, pH 7.4; and β-Ala-Gly-Arg-pNA-acetate (1 mM) in 50 mM Tris, 100 mM NaCl, 5 mM CaCl₂, pH 7.4. Factor Xa substrate was CH₃-D-CHA-Gly-Arg-pNA-AcOH (0.5 mM) in 50 mM Tris, 100 mM NaCl, 5 mM CaCl₂, pH 7.4. Plates were sealed with tape and parafilm to avoid evaporation and incubated at +37° C. Absorbance was measured at 405 nm using Microplate Reader (BP-808, Biohit). Measured sample values were compared to a standard curve made using thrombin from Terumo. Only thrombin substrate Sar-Pro-Arg-pNA gave weak positive results with serum samples after 18 hours incubation. The results did not correlate with the presence of CNPs, based upon Capture ELISA results. Thrombin substrates Bx-Phe-Val-Arg-pNA HCl and -Ala-Gly-Arg-pNA-acetate failed to give positive signals. Factor Xa substrate CH₃-D-CHA-Gly-Arg-pNA-AcOH gave weak positive results for serum samples after 18 hours incubation. Results did not correlate with the presence of CNPs. Thus, the results indicate only non-specific binding of thrombin and Factor Xa activity to ELISA plate which was present only in serum samples. Therefore, the CNP-bound antigens must have been in an inactive form, as is expected in blood samples of healthy people.

x. Immunohistochemical Staining for Antigen Pattern Analysis

Paraffin-embedded arterial tissue blocks representing various forms of severe atherosclerotic lesions were obtained from commercial sources (Clinomics BioSciences, Inc., Pittsfield Mass. 01201. Tissue samples were collected from New York area and processed under Institutional Review Board permit). Thin sections were cut using standard techniques. Sections were deparaffinized without decalcification and stained with monoclonal antibodies for antigen pattern analysis mapping calcification defense proteins, clotting factors and CNPs. The staining protocol was tailored for each antibody, see Table 3. PowerVision+™Poly-HRP IHC kit (ImmunoVision Technologies, Brisbane Calif. 94005) was used according to manufacturer's instructions. Color was developed using 3,3′-diaminobenzidine substrate. Sections were counterstained using Mayer's Hematoxylin (Reagena, Siiilinjärvi, Finland), mounted and staining results were evaluated with light microscopy. Calcium deposits were stained with von Kossa staining. Dehydrated slides were immersed into 5% silver nitrate (BDH) solution for 1 hour under 100 W lamp. Slides were rinsed shortly with distilled water and further immersed into 5% sodium thiosulphate (Merck) for 2 minutes. Slides were washed three times with distilled water before staining with Kernetchrot solution (Reagena) for 5 minutes. Slides were rinsed with distilled water, dehydrated and mounted with Depex (BDH). Samples were microscoped with Nikon FXA microscope. Antigen patterns in soft plaque and hard plaque were compared. A specific pattern was observed that involved strongest intensity of staining in calcifications, followed by focal necrotic areas, and individual areas in media and intima layers. The pattern was comparable for all studied G1a protein antigens and CNPs. Patterns of Factor XIIIA and tissue factor showed a more universal presence, albeit also indicated accumulations of these antigens in the atherosclerotic lesions. Stainings carried out omitting primary antibody were negative. Calcification staining with von Kossa method matched with large macroscopic calcifications and some of the microscopic areas where CNP and G1a protein stainings revealed positivity. This can be interpreted to mean that although the tissue staining pattern was comparable, the sensitivity of von Kossa staining is inferior to immunostainings in detecting CNPs and nanoscopic calcifications.

2. Results

This example various forms of apatite were studied, including inorganic synthetic (Poser and Price, J. Biol. Chem. 254, 43 (1979)) and organic form built on calcifying nano-particles, referred to previously as nanobacteria (Kajander and [symbol] iftçioglu, Proc. Natl. Acad. Sci. USA 95, 8274 (1998)). Hydroxyapatite formation includes several metastable calcium phosphate intermediate phases (Nancollas, Pure & Appl. Chem, 11, 1673 (1992)). Brushite (dicalcium phosphate dihydrate) and octacalcium phosphate are considered initial phases that are transformed to hydroxyapatite, which is the most insoluble calcium phosphate mineral phase forming under neutral or basic conditions (Johnsson and Nancollas, Crit. Rev. Oral, Biol. Med. 3, 61 (1992)). Basic calcium phosphate (BCP) is a term that includes various calcium phosphate minerals, i.e. hydroxyapatite, carbonate-apatite, octacalcium phosphate and brushite. In this example the general name apatite is used for the calcium phosphate mineral.

Synthetic colloidal apatite was used as a control while performing acute toxicity studies for calcifying nano-particles (CNPs). Surprisingly, it was found that both iv injected apatite and CNPs caused ischemia-type tissue damage in the kidneys of rats. The pathognomic feature in ischemia-reperfusion kidney damage is that glomeruli are saved whereas tubuli die (Park et al., Am. J. Physiol. Renal Physiol. 282, F352 (2002)). The kidney damage was dose-dependent, and did not occur when two microliter or less apatite was injected. Control animals receiving only phosphate buffered saline (PBS) did not show histological changes in kidneys. There were also signs of thrombotic events in large blood vessels and cardiac chamber walls.

To verify thrombotic events after iv injection, bio-distribution of ^{99m}Tc-labeled colloidal apatite and CNPs was reexamined by using Single Photon Emission Computed Tomography (SPECT) after iv-injection in rabbits (Akerman et al., Proc SPIE Int Soc Opt Eng 3111, 436 (1997)). Thrombosis was observed in left pulmonary artery starting within one minute of the injection and was stable and could still be detected 48 h after injection.

i. How was Thrombosis Activated?

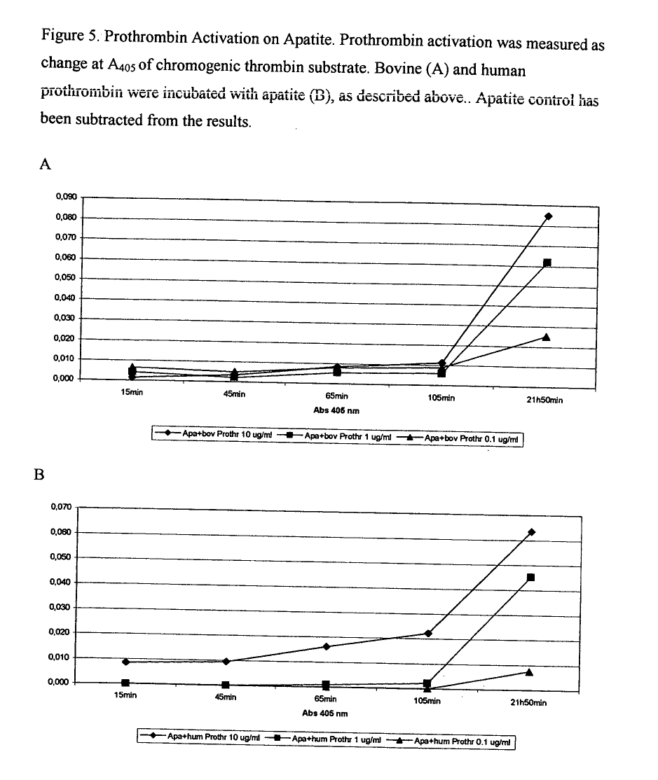

Standard blood coagulation tests (e.g., activated partial thromboplastin time, prothrombin time) were inappropriate to measure calcium phosphate mineral surface-mediated clotting because those tests require use of anticoagulants, which would interact with an apatite surface. Counteracting the anticoagulants with high calcium chloride concentrations, as is required in the tests, creates non-physiological competition for binding between free calcium (tens of times higher than the physiological) and calcium phosphate surface. Furthermore, the apatite surface would be modified by a solution high in calcium, forming other forms of calcium minerals on the surface (e.g., octacalcium phosphate) (Boskey, J. Phys. Chem. 93, 1628 (1989)). Apatite is stable under physiological calcium and phosphate concentrations. Therefore, to study the effects of apatite on clotting, a whole blood clotting slide test was developed. In this test, plain objective glass, or objective glass coated with various forms of apatite, or test drugs, were used as test platforms. 200 μL of freshly collected human blood was applied on the slides, which were tilted ±30°, 15 tilts per minute, at room temperature. Clotting time was established at the time when droplet contents stopped moving. The test indicated that clotting was two times faster on apatite coating compared to the control slide. CNP coating also decreased clotting time significantly (FIG. 6; Table 1). The method was controlled by using EDTA or citrate plasma samples, which never clotted, even when exposed to apatite coated test platforms. Calcium EDTA and a small concentration of the calcium binding drug etidronate did not affect the clotting time. Therefore the test appropriately measured clotting triggered by a foreign surface, glass. It was surprising that the apatite surface was superior at inducing clotting over the untreated glass, the traditionally used foreign surface in clotting tests.

ii. How Does Apatite Cause Clotting?

Calcium is a major player throughout the clotting process. The extrinsic clotting pathway is activated by tissue factor, which is a 40 kD membrane-spanning protein expressed normally by almost all cells, except the endothelium. Endothelial damage exposes tissue factor, which binds and allosterically activates a serine protease, factor VIIa (FVIIa), in the presence of calcium. This complex then proteolytically activates two serine protease zymogens, factor IX (FIX) and factor X (FX) (Banner et al., Nature 380, 41 (1996)), again in the presence of calcium, resulting in formation of factor Xa (FXa), which splits prothrombin to thrombin, again in the presence of calcium, which is the final coagulation executor proteolytically insolublizing fibrinogen as fibrin. The clot is further stabilized by cross-linking by factor XIIIa (FXIIIa) (activated by thrombin in the presence of calcium). The intrinsic pathway commences upon exposure to a foreign negatively charged surface, activating calcium-dependent conformational changes of clotting factors resulting in binding to a platelet or other phospholipid membrane, and leading to an activation-amplification cascade which eventually activates FX resulting in thrombin release.

Proteomics analysis revealed prothrombinase complex on apatite surface together with players of complement, antibodies and protease inhibitors. Although the use of serum to test clotting factors is not preferred, this proved the ability of apatite surface to bind clotting factors and provided information about what proteins can bind in biological situations, for instance on CNPs.

The same proteomics method was not preferred for CNPs because 1-D or 2-D electrophoresis can not be run without extracting proteins from the particles, which is difficult. Difficulty in extracting proteins form CNPs indicates that the proteins on CNPs are cross-linked. Therefore, the surface protein patterns of CNPs were mapped using Surface Antigen Pattern Immuno-Assay (SAPIA; FIG. 1), which is an example of a specific embodiment of the disclosed method of detecting proteins on calcifying nano-particles. In this technique specific antibodies directed against clotting and anti-mineralization proteins were used (the 5/2 capture antibody from the Nanocapture Elisa kit can be used to form the standard curve for any of the assays. The results can be used for calculation of algorithms for specificy disease diagnosis). SAPIA profiles of CNPs using plasma and serum samples were practically identical (FIG. 3). These results indicated that serum samples can be used for the test. The results also indicated that particles with this specific antigen surface pattern can be isolated from human blood without any culturing steps. SAPIA results were stable after freeze-thawing, detergent (Tween20), EDTA or citrate application. Evidence was found that the detected proteins are cross-linked (very little protein released by SDS boiling). The stability of CNPs makes them amenable to surface antigen mapping with SAPIA technique which involves extended step-wise incubations separated by numerous washings before the detection. This feature of CNPs also allows the use of harsh treatments, when useful or desired, in other assays and detection methods.

SAPIA indicated that clotting factors V, VII, IX, X, tissue factor-FVIIa complex, fibrin, fibrinogen, FXIIIa, fragments of factor II, thrombin and prothrombin Fragment 1, but not prothrombin Fragment 2 are on CNPs (Tables 2 and 3; FIG. 2). Both matrix GLA-protein and osteocalcin were present on CNPs as well. The pattern of these factors was positively correlated with results using the Nanocapture kit with a high confidence level, indicating that the particles are composed of these surface antigens. SAPIA did not give positive results with capture antibodies specific for fibronectin (FIG. 3) nor for human serum albumin, D-Dimer or NF-kB. The results prove that the particle surface contains players of the intrinsic and the extrinsic pathway (Table 2). Much of the CNP prothrombin has been activated to release thrombin and prothrombin Fragment 1. Prothrombin fragment 1 has 10 G1a residues whereas fragment 2 has none, which can explain why fragment 2 is released. However, it was surprising that the thrombin is retained in the particle. There may be mechanism(s) to retain it, such as crosslinking, or complex formation. For example, thrombin is known to make a complex with FXIII and fibrin (Ariens et al., Blood 100, 743 (2002)), which were also found on the particle.

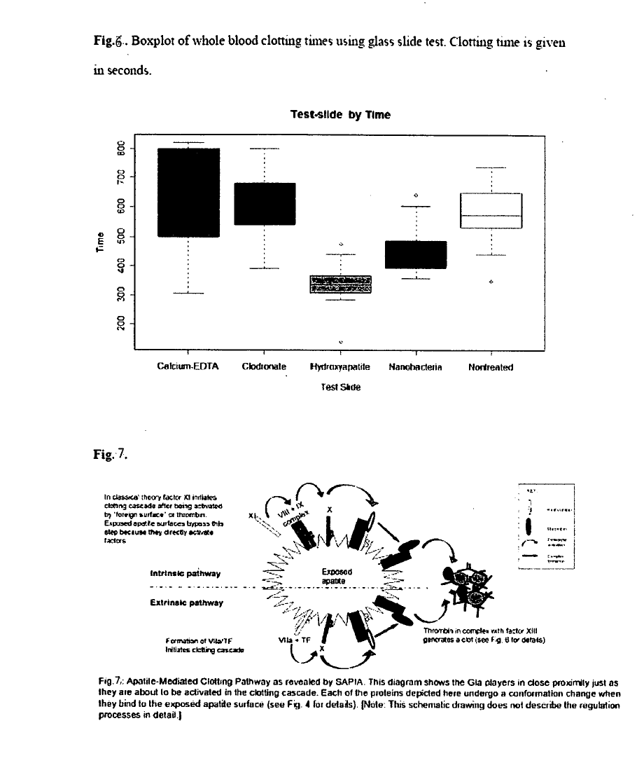

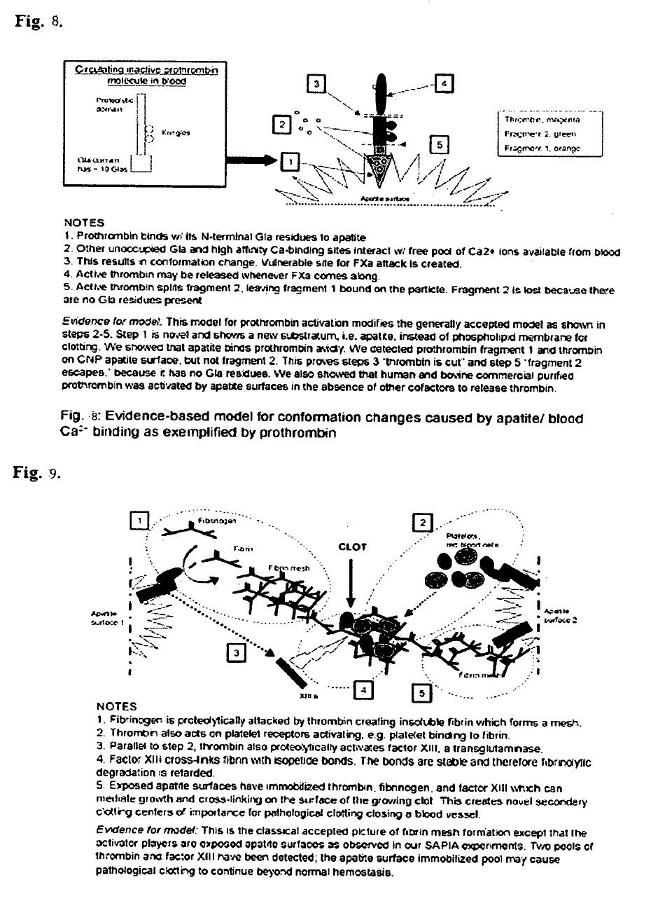

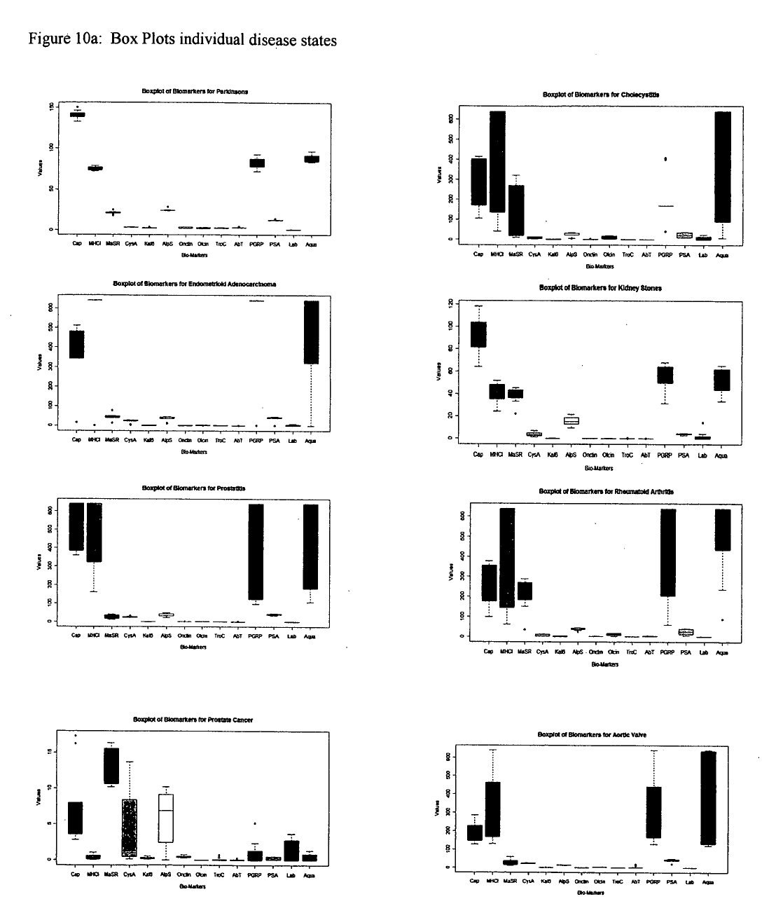

It was discovered that apatite binds clotting factors and their activators, concentrating them in close proximity, thus providing the necessary players for clotting on a suitable substratum (FIGS. 7-9). G1a residues in the G1a domain are known to bind to apatite. Free blood calcium completes the activation by binding to the rest of G1a-residues (FIG. 8). Interaction of prothrombin with hydroxyapatite mineral has been shown by Romberg (Romberg et al., Arterioscler. Thromb. Vasc. Biol. 18, 33 (1998)) to be comparable to anti-mineralization G1a-proteins. The clotting cascade has G1a-containing factors II, VII, IX, and X, and their levels have been linked to hypercoagulability and as risk factors for atherosclerosis and its thrombotic complications (Xu et al., Arterioscler. Thromb. Vasc. Biol. 18, 33 (1998); Carlsson et al., Eur. J. Biochem 270, 2576 (2003)). Calcium phosphate, the key element in apatite, is a normal body constituent, therefore cannot be regarded as foreign surface that activates the intrinsic pathway of clotting. The key player in the extrinsic pathway, tissue factor, is not a calcium binding protein but might act as a player on the apatite after complex with FVIIa (Table 2; FIG. 7).

To find out whether particle-bound thrombin was active, chromogenic thrombin substrate incubations were made after capture of the CNPs. Very slight conversion of thrombin substrate was observed after 3 hours incubation at 20° C. in the most positive samples, but this could have been due to other proteolytic activity on CNPs. Therefore, most if not all of particle immunologically detected thrombin must have been inactive, as was expected, because active thrombin would otherwise lead to dire consequences. The body has an efficient anti-thrombin system to inactivate the thrombin. SAPIA for detecting Anti-thrombin III gave only 2/16 high positive results (for the samples with the highest Nanocapture results). The test may have failed to detect less positive cases due to the use of an antibody that binds to an epitope known to block the thrombin-anti-thrombin complex formation, which means that its epitope was inside the inactive complex. Factor Xa activity chromogenic substrate test revealed no activity. This factor is also inactivated by anti-thrombin.

It was also tested whether non-enzymatic activation of thrombin could take place on apatite. The results presented in this example indicate that a tiny amount of thrombin activity, measurable with the chromogenic substrate, was generated on the apatite but not on the ELISA plate surface exposed with purified human or bovine prothrombin.

Using SAPIA, matrix G1a-protein and osteocalcin were identified on the CNPs. The presence of calcification-defense G1a proteins and blood clotting factors of the extrinsic and intrinsic pathways were identified on the same particle. To determine if the two classes of G1a proteins could be associated with calcification in atherosclerotic lesions in humans, immunohistochemical stainings on human atherosclerotic lesions were made. Immunohistochemical staining showed CNPs were concentrated in both soft plaque and overt calcifications or hard plaque areas. The same type of localization was found for several members of both G1a protein families. It is noteworthy that von Kossa staining could detect only large calcific areas whereas the CNPs were found as almost nanoscopic calcifications.

3. Discussion

The results in this example show that apatite surfaces lead to rapid activation of the clotting cascade. Among other implications, this provides support for long-established clinical evidence suggesting that calcified lesions participate in formation of atherothrombi. It was proven that apatite surfaces simultaneously bind multiple G1a proteins, some of which are clotting proteins. This creates a platform for a clotting mechanism that combines both intrinsic and extrinsic factors to the common FX and prothrombin activation. This clotting mechanism is shown in FIGS. 7-9. The diagram depicts a novel platform, formation of complexes and activation of a clotting cascade on apatite surfaces. It was also shown that apatite itself can contribute to conformational changes leading to activation of prothrombin on apatite surfaces to release active thrombin. This non-enzymatic activation was much less rapid without the added clotting cascade players, yet proves the essential role that apatite plays. Prothrombin activation involves initial reactions with calcium, followed by a membrane prothrombinase complex formation leading to a thrombin release (Borowski et al., J. Biol. Chem. 261, 14969 (1986)). The players in this complex and their activators were shown in this example to be bound on apatite surfaces. It was also shown that the apatite-mediated clotting was twice as fast as clotting mediated by contact with a foreign surface, in this case glass, activating the intrinsic pathway.

The apatite-mediated clotting cascade, as with other clotting cascades, would have to be meticulously controlled by anti-thrombin, Protein S and C, heparin and other anti-clotting mechanisms and fibrinolytic systems to maintain a fine balance between activation and inhibition. Tissue factor found on CNPs shows that apatite particles can activate clotting using extrinsic pathway players. This process could be controlled by inhibitors, for example, by tissue factor inhibitor pathway (TFIP), which is likely since CNPs were not more active than apatite, which lacked the presence of the tissue factor. However, for the reasons shown above, processes required to control apatite-mediated clotting break down, leading to massive thrombosis.

This example shows for two forms of apatite that sudden circulatory exposure leads to thrombotic events, indicating that exposure of blood to apatite can have catastrophic results. Thrombosis was found when blood in a vessel was suddenly exposed to apatite pellet (colloidal) volume in excess of two microliters. Apatite exposure of this magnitude could take place as a consequence of, for example, bone fracture, rupture of vulnerable plaque revealing pathological vascular calcification, or in any situation where circulatory apatite particle counts would become locally high, for example, after rupture of a cyst filled with them.

Apatite-mediated clotting can have an important physiological function in bone physiology. Large bones have cancellous surface compartments with a diameter larger than largest blood vessels. Thus bone fracture often leads to clots up to 10 centimeters in diameter that must be made relatively rapidly to prevent the victim from bleeding to death. Exposed apatite could serve as the platform, providing booster power for clotting, since the hollow bone cannot reduce its diameter as damaged blood vessels do via vasoconstriction, and the bone has few tissue factor sources. Bone trabeculae are covered with only a monocellular layer, endostium, and the cortical bone has very low cell density (no subendothelial cells available with cell tissue factor carrying membranes as present in other tissues). It had not been clear how tissue factor-mediated clotting could take place in bone, but based on the results in this example it can be seen that the exposed bone could allow apatite-mediated clotting. Intriguingly, bone contains significant amounts of clotting G1a proteins. Those proteins are present at 1-2% level of the non-collagen proteins in bone They could act with the bone G1a-protein osteocalcin, which was also found on CNPs, to control bone mineralization and/or provide protection against bleeding after bone fracture, where large areas of calcified surface are exposed. G1a-proteins are also found in kidney stones, suggesting a role in stone formation via both mineralization and thrombin production via thrombotic events or other mechanisms. Prothrombin F1 is the most common protein associated with kidney stones, and thrombin has been detected in urine in kidney diseases. Thrombogenic mechanisms have been proposed for kidney stone formation (Stoller et al., J. Urol. 171, 1920 (2004)).

There is a very high incidence of calcifying nano-particles in disease processes known to be associated with calcification/thrombosis, for example, 97.5% associated with carotid stenosis, whereas only 10% association in Crohn's disease. Furthermore, the faster whole blood clotting time observed with calcium phosphate (bio)films supports the hypothesis from cardiology that rupture of unstable plaque endothelium exposing calcific plaque can cause clotting and lead to thrombosis and myocardial infarction or stroke. CNPs were shown to be present in atherosclerotic calcification and in soft plaque as nanoscopic calcific particles. This is consistent with a pathological role for any sized calcification in the vasculature. Calcification can be the driving force for atherothrombosis. The connection between apatite particles in the wrong part(s) of the body and inflammation and immunological activation has been shown (Morgan et al., Arthritis Rheum. 50, 1642 (2004); Nadra et al., Circ. Res. 96, 1248 (2005)).

Immunohistochemical stainings revealed the accumulation of clotting and anti-mineralization G1a-proteins on the CNPs. This evidence directly shows that G1a proteins have particular accumulations in atherosclerotic lesions associated with calcifications of all sizes. Thus, calcification is linked to both clotting factors and anti-mineralization G1a-proteins. Both clotting factors and anti-mineralization G1a-proteins have been separately shown to co-localize with calcification in atheromas and similar types of lesions (Bini et al., Arterioscler. Tromb. Vasc. Biol. 19, 1852 (1999); Mullins et al., FASEB J. 14, 835 (2000); McKee Nature, but not in the same lesions prior to the results in this example. Since CNPs are detectable just below the endothelium, they can contribute to thrombotic clotting together with the circulating CNPs when the endothelium lining is damaged. The results in this example indicate a role for an apatite-mediated clotting system in thrombotic events.

Studies on thrombogenicity of biomaterials have examined heparin stabilized apatite, or heparinized animals. Since heparin is an anticoagulant, such studies do not reveal thrombotic potential adequately. Thus, biocompatible materials may not be hemocompatible. Apatite-coated implants are widely used due to their bone biocompatibility. Many new medical applications for apatite have been proposed including drug delivery systems to blood, lung airways, or tissue; as a vaccine adjuvant; a vehicle for DNA transfer; and even as stent material for blood vessels. However, the effects of implants or injected apatite outside of bone and teeth have not been widely studied. Biocompatibility studies for calcium phosphate mineral particles or surfaces do not seem to have reported thrombotic events. Aoki et al reported vascular collapse as the cause of death in rats injected i.v. with colloidal apatite. Although Aoki reported severe hypoxia and elevation of infarct enzymes, above a threshold dose, the intravenous administration to humans was suggested to be feasible while using dosage less than LD50 for rats (Aoki et al., J. Mater. Sci. Mater. Med. 11, 67 (2000)). However, thrombi in rats injected with apatite i.v. were observed as descrubed in this example. Therefore, the biocompatibility of apatite, when exposed to circulation, is now seriously in question. This does not suggest that apatite use in implants should be discontinued, but rather that it must be separated from the blood in biocompatible ways with, for example resilient coatings, just as the body separates natural bone from blood via endostium. Thus, disclosed herein are compositions comprising apatite and a coating material, where, for example, the coating material limits exposure of the blood of a subject when the composition is in a subject.

The contrast between results in this example and apatite biocompatibility published earlier may be due to the ISO 10993-4. It requires the use of citrate or hirudin blood, or plasma and allows their application on implant materials while performing hemocompatibility testing (Seyfert et al., Biomolecular Engineering 19, 91 (2002)). The results in this example indicate that ISO 10993-4 required conditions cannot be used to detect blood clotting on apatite. The following improvements in biomaterial testing were devised: (1) Complementing ISO 10993-4 with tests for whole blood clotting in the absence of anticoagulants; and (2) Stents, catheters and materials exposed to circulation should repel calcific biofilm formation, because calcifying particles have been found to bind and coat such surfaces (Kajander et al., NASA/CP-2002-210786, 51 (2002)). Thus, disclosed herein is a method of testing biocompatibility comprising testing blood coagulation in the absence of anticoagulants and a method of testing materials that will be exposed to circulating blood for formation of calcific biofilm formation. Subjects in which pathological thrombosis can occur via apatite-mediated clotting are useful targets for these and other method disclosed herein. Such subject include (1) Patients with vulnerable plaque rupture exposing atheroma calcification; (2) Patients undergoing angioplasty or heart-lung machine perfusion; (3) Patients with massive bone fractures or dislocated implants releasing potentially apatite particles; (4) Patients with implants, catheters, wires or stents subject to calcium encrustation; (5) Cancer patients with soft tissue calcification; and (6) Healthy or sick people with CNPs in their blood or positive calcification scores in arteries. Such people in the last category can be identified using the disclosed compositions and methods.

This example describes a newly discovered pathophysiological mechanism linking pathological calcification to thrombosis. Blood anti-calcification G1a-proteins and G1a-clotting factor proteins were shown to bind to calcium phosphate surfaces creating a novel clotting mechanism capable of causing thrombosis where blood is in contact with apatite or CNPs. This was shown by detecting thrombosis after IV injections of apatite and CNPs in vivo in rats and rabbits, leading to thrombotic events, including ischemia-reperfusion damage. A whole blood coagulation slide test was developed to measure effects of various surfaces, including apatite and CNP, on blood clotting in vitro. Avid binding of G1a-clotting factors to apatite was detected by proteomics. A novel SAPIA test was developed to demonstrate the pattern of clotting factors and their activators on the surface of CNPs isolated from human plasma or serum. The apatite prothrombin interaction resulted in a small amount of thrombin activity detected by chromogenic substrate, despite the absence of other clotting factors.

### B. Example 2

The SAPIA method described in Example 1 was utilized to detect CNP components in 16 serum samples. Tables 11 and 12 illustrate the results of SAPIA testing. Table 11 shows raw absorbance data in the upper half of the table for 97 proteins and components measured in 16 human serum pools. The lower half illustrates units per ml. The pools were obtained by mixing the serum from 1-5 donors for each pool, pooled according to capture ELISA results that showed similar antigen levels.

The results show, with few exceptions, that each protein or component was detected on CNPs. Significant patterns are realized via intpretation of the data. Observations showed that certain proteins and/or components exhibit high and low values. Trends were realized and, for example in Table 11, different patterns are shown by anti-thrombin and anti-osteocalcin. At least 6-12 patterns are able to be visualized (qualitatively) by those skilled in the art.

Table 12 shows the statistical analysis as generated from the raw data of Table 11. The table shows correlation between the markers (100×100). Correlation coefficients greater than 0.5 indicate positive correlation (with low p values) and those values approaching 0.0 indicate a negative correlation. Therefore, statistical review via the generation of, for example, of box plots or scatter plots enables one skilled in the art to visualize data patterns that may be useful in the assessment, diagnosis, and therapeutic selection for certain diseases and/or conditions. Various algorithmic methods may be applied, for example, by multiplying, dividing, addition, or subtraction for various antigen values. These algorithms may be used in the diagnosis of diseases and/or conditions.

Data may be further analyzed via more sophisticated techniques, for instance, cluster analysis, neural network, or multivariate logistic regression techniques.

Cluster analysis is a multivariate statistical technique which assesses the similarities between units or assemblages, based on the occurrence or non-occurrence of specific artifact types or other components within them.

Neural networks are a well-established technology for solving prediction and classification problems, using training and testing data to build a model. The data involves historical data sets containing input variables, or data fields, which correspond to an output. The network uses the training data to “learn” the solution to the problem by example. Since the network learns in this way, no complex models need to be created. Also, it is not necessary for your data to be complete or show a clear trend—neural networks can still converge to a solution under these conditions.

Logistic regression is part of a category of statistical models called generalized linear models. This broad class of models includes ordinary regression and ANOVA, as well as multivariate statistics such as ANCOVA and loglinear regression. An excellent treatment of generalized linear models is presented in Agresti (1996).

Logistic regression allows one to predict a discrete outcome, such as group membership, from a set of variables that may be continuous, discrete, dichotomous, or a mix of any of these. Generally, the dependent or response variable is dichotomous, such as presence/absence or success/failure. Discriminant analysis is also used to predict group membership with only two groups. However, discriminant analysis can only be used with continuous independent variables. Thus, in instances where the independent variables are a categorical, or a mix of continuous and categorical, logistic regression is preferred.

These selected markers (biomarkers) are well known in the art to be indicative of or risk factors for disease.

### C. Example 3

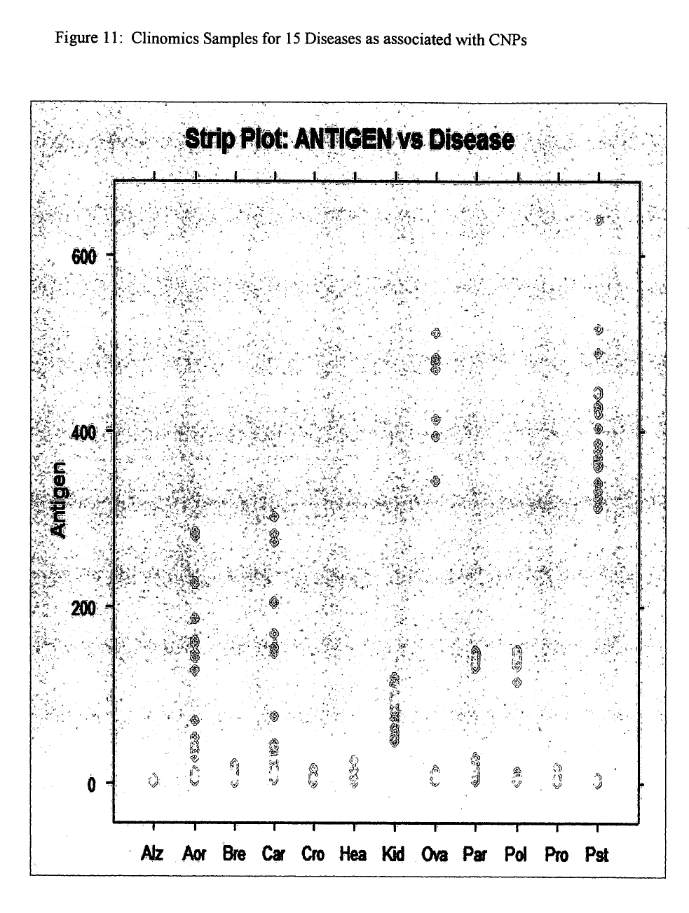

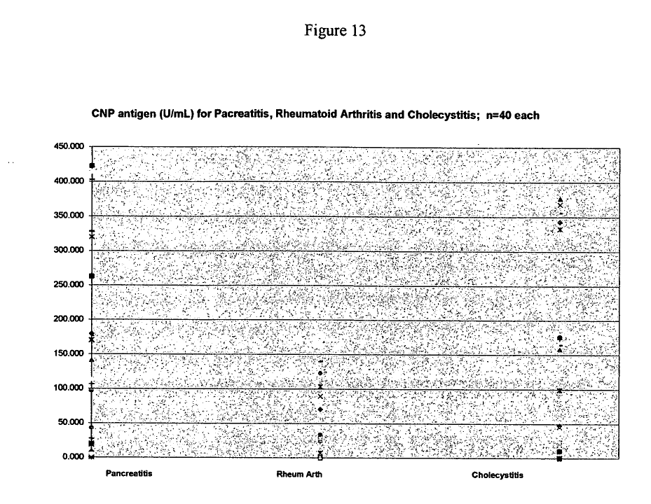

Disease specific sera was obtained from Clinomics Bioscience Inc. for 15 diseases, n=40 per disease. Nano capture results are shown in FIGS. 11 and 13. The data indicated that CNPs were detectable in each disease, however at quite different percentages and levels. To understand the meaning of the biomarkers present in said CNPs, 10 samples showing the highest values for CNPs were selected for further analysis of biomarkers on the CNPs.

The data show clearly evident disease patterns easily recognized to the trained eyes of those skilled in the art. Especially prominent patterns were seen in prostatitis and prostate cancer. The establishment of these ratios and scatter graphs (see FIGS. 11 and 13 respectively) offer substantial improvements to the diagnosing of these diseases.

In prostate cancer and prostatitis, the most important biomarkers are the presence or absence or MHC-1, Macrophage Scavenger Receptor, Osteocalcin, PGRP-1, PSA, Aquaporin-4. The results may be better analyzed by comparison of specific marker values to the capture results. In prostate cancer, the ratio of marker Macrophage 1:1.5 to (or and approximate 30 fold difference) capture, whereas in prostatitis the ratio is 1:0.5.

Similarly, the ratio result (prostate cancer) MHC-1 to capture is about 5% whereas the ratio in Prostatitis is almost 1.0 or a 20 fold difference. Significantly, Osteocalcin shows importance as either a presence and absence value as it is not present in Cancer. For PGRP-1, the difference is approximately 0.15 in Prostate Cancer and 0.8 in prostatitis, meaning approximately 5.5 fold.

PSA shows a value of approximately 0.076 in Prostatitis and 0.03 in prostate cancer, or approximately 2 fold differences. This is a very small factor in favor of prostate cancer. In TG2 (labvision) the difference is 0.0009 in Prostatitis and approximately 0.15 in prostate cancer, a difference of approximately 166 fold.

For Aquaporin, values show 0.8 in Prostatitis and 0.05 in prostate cancer, approximately a 20 fold increase in prostatitis.

Therefore, levels of these different markers are very important in the diagnosis of disease. For example, analysis via custer analysis that may involved sophisticated methods such as multivariate logistic regression.

In Psammoma Endometrioid adenocarecenoma, the most important biomarkers that are present or absent are MHC-1, Cystatin A, osteocalcin, PGRP-1 Beeta, PSA, Labvisoin TG-2, Aquaporin-4. Psammoma Endometrioid adenocarecenoma had 8 groups with extremely high calcification that may be, for instance, easily separated by the correlation of the presence or absence MSR and PGRP-1 Beeta and Aquaporin-4. Notable is that some normal positive high value had high PSA.

Therefore, disease specific marker tests results indicate that since the measurements were made using human blood samples different patterns of antigens on CNP may be explained only by assuming that those markers were bound on the surface of the CNP at the specific location of the pathological process. Therefore, these markers as associated with the CNP may be used to diagnose pathological processes, diseases, and ongoing processes leading to pathological problems (risk analysis and therapy follow up). This is due to the fact that different tissue and cells contain different (and the same) types of specific markers. It is well known that markers for diseases can be present YEARS before the onset of disease. Therefore these biomarkers can detectable prior to clinical diagnosis of disease and may be used as risk factor analysis or early detection of diseases including, but are not limited to, for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

### D. Example 4

Evidence of Various Type of CNP

Biomarker patterns found indicate that various types of CNPs exist in the body of animal. The presence of different “types” has been verified by testing biodistribution in rats. Test results showed very different bio-distributions for the CNPs. It was determined that CNPs vary depending upon where they are harvested.

For example, CNPs taken from hosts and passage several times in various growth media, then injected into the tails of rats still showed specific characteristics depending upon original location of harvesting.

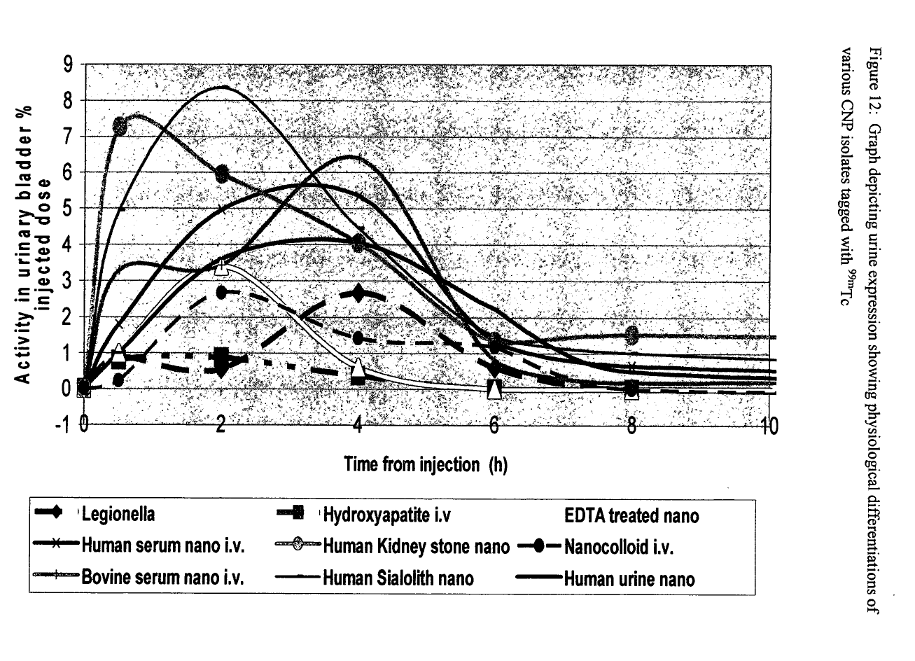

FIG. 12 shows the excretion in urine from a RAT. The excretion kinetics in the urine were very different. The most pronounced differentiation was shown with the Kindey stone isolate.

Table 8 is a list of some proteins that can be on CNPs. Table 9 is a list of proteins and compounds that can be associated with CNPs and proteins on CNPs. Table 10 shows calculated unit per ml data from 8 diseases using 14 markers and 10 patient samples for each disease. Table 11 shows the use of SAPIA technique to map Proteins associated with CNPS (Raw Data plus units per ml data). Table 12 shows a table on correlation on SAPIA results for various proteins and antigens on CNPs (coefficients and significances).

LENGTHY TABLE REFERENCED HERE
US20070134814A1-20070614-T00001
Please refer to the end of the specification for access instructions.

LENGTHY TABLE REFERENCED HERE
US20070134814A1-20070614-T00002
Please refer to the end of the specification for access instructions.

LENGTHY TABLE REFERENCED HERE
US20070134814A1-20070614-T00003
Please refer to the end of the specification for access instructions.

LENGTHY TABLE REFERENCED HERE
US20070134814A1-20070614-T00004
Please refer to the end of the specification for access instructions.

LENGTHY TABLE REFERENCED HERE
US20070134814A1-20070614-T00005
Please refer to the end of the specification for access instructions.

LENGTHY TABLE REFERENCED HERE
US20070134814A1-20070614-T00006
Please refer to the end of the specification for access instructions.

LENGTHY TABLE REFERENCED HERE
US20070134814A1-20070614-T00007
Please refer to the end of the specification for access instructions.

LENGTHY TABLE REFERENCED HERE
US20070134814A1-20070614-T00008
Please refer to the end of the specification for access instructions.

LENGTHY TABLE REFERENCED HERE
US20070134814A1-20070614-T00009
Please refer to the end of the specification for access instructions.

LENGTHY TABLE REFERENCED HERE
US20070134814A1-20070614-T00010
Please refer to the end of the specification for access instructions.

LENGTHY TABLE REFERENCED HERE
US20070134814A1-20070614-T00011
Please refer to the end of the specification for access instructions.

LENGTHY TABLE REFERENCED HERE
US20070134814A1-20070614-T00012
Please refer to the end of the specification for access instructions.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a calcifying nano-particle” includes a plurality of such calcifying nano-particles, reference to “the calcifying nano-particle” is a reference to one or more calcifying nano-particles and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

LENGTHY TABLE
The patent application contains a lengthy table section. A copy of the table is available in electronic form from the USPTO web site (http://seqdata.uspto.gov/?pageRequest=docDetail&DocID=US20070134814A1). An electronic copy of the table will also be available from the USPTO upon request and payment of the fee set forth in 37 CFR 1.19(b)(3).

We claim:

1. A method for detecting calcifying nano-particles, the method comprising
detecting calcifying nano-particles by detecting one or more proteins or components on the calcifying nano-particle.

2. The method of claim 1, wherein the calcifying nano-particles are detected by detecting one or more of the proteins selected from the group consisting of the proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

3. The method of claim 1, wherein the calcifying nano-particles are detected by detecting two or more proteins or components on the calcifying nano-particle.

4. The method of claim 1, wherein the calcifying nano-particles are detected by detecting one or more proteins or components with a GLA-containing domain.

5. The method of claim 1, wherein the calcifying nano-particles are detected by detecting one or more proteins or components with a calcium binding domain.

6. The method of claim 1, wherein the calcifying nano-particle is captured, identified, or both prior to, simultaneous with, or following detection of one or more of the proteins or components.

7. The method of claim 6, wherein capture or identification of the calcifying nano-particle indicates that the detected proteins or components are on the calcifying nano-particle.

8. The method of claim 6, wherein the calcifying nano-particle is captured by binding at least one compound to one or more of the proteins or components, wherein the compound is or becomes immobilized.

9. The method of claim 6, wherein the calcifying nano-particle is identified by binding at least one compound to one or more of the proteins or components, wherein the calcifying nano-particle is separated based on the compound.

10. The method of claim 9, wherein the calcifying nano-particle is separated by fluorescence activated sorting.

11. The method of claim 1, wherein one or more of the proteins are detected by binding at least one compound to the protein and detecting the bound compound.

12. The method of claim 11, wherein detection of two or more bound compounds indicates that the proteins to which the compounds are bound are on the calcifying nano-particle.

13. The method of claim 12, wherein the two or more compounds are detected in the same location or at the same time.

14. The method of claim 11, wherein at least one of the compounds is an antibody, wherein the antibody is specific for the protein.

15. The method of claim 1, wherein the calcifying nano-particles comprise calcium phosphate and one or more of the proteins.

16. The method of claim 1, wherein the proteins are detected by detecting any combination of 100 or fewer of the proteins selected from the group consisting of proteins with a GLA-containing domain, clotting factor V, clotting factor VII, clotting factor IX, clotting factor X, tissue factor-clotting factor VIIa complex, fibrin, fibrinogen, factor XIIIa, fragments of factor II, thrombin, prothrombin Fragment 1, matrix GLA-protein and osteocalcin on the calcifying nano-particle.

17. The method of claim 16, wherein the proteins are detected by detecting any combination of 75 or fewer of the proteins.

18. The method of claim 17, wherein the proteins are detected by detecting any combination of 50 or fewer of the proteins.

19. The method of claim 18, wherein the proteins are detected by detecting any combination of 10 or fewer of the proteins.

20. The method of claim 16, wherein the combination of proteins is detected in the same assay.

21. The method of claim 16, wherein the combination of proteins is detected simultaneously.

22. The method of claim 16, wherein the combination of proteins is detected on the same calcifying nano-particle.

23. The method of claim 16, wherein the combination of proteins is detected on or within the same device.

24. The method of claim 16, wherein the combination of proteins detected constitutes a pattern of proteins.

25. The method of claim 24, wherein the pattern indicates or identifies a disease or condition, a risk of a disease or condition, the severity of a disease or condition, or a combination.

26. The method of claim 24, wherein the pattern indicates or identifies a treatment to inhibit, remove or prevent the calcifying nano-particles.

27. The method of claim 24, wherein the pattern identifies the type of calcifying nano-particles detected.

28. The method of claim 1, wherein the proteins are detected by detecting the presence or absence of any combination of 10 or fewer of the proteins selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type 1, anti-Thrombin, Protein S, BAFF on the calcifying nano-particle.

29. The method of claim 28, wherein the pattern of the presence or absence of the proteins indicates or identifies a disease or condition, a risk of a disease or condition, the severity of a disease or condition, or a combination.

30. The method of claim 28, wherein the pattern of the presence or absence of the proteins indicates or identifies a treatment to inhibit, remove or prevent the calcifying nano-particles.

31. The method of claim 28, wherein the pattern of the presence or absence of the proteins identifies the type of calcifying nano-particles detected.

32. The method of claim 28, wherein the presence of one or more of the proteins indicates or identifies a disease or condition, a risk of a disease or condition, the severity of a disease or condition, or a combination.

33. The method of claim 28, wherein the presence of one or more of the proteins indicates or identifies a treatment to inhibit, remove or prevent the calcifying nano-particles.

34. The method of claim 28, wherein the presence of one or more of the proteins identifies the type of calcifying nano-particles detected.

35. The method of claim 28, wherein the absence of one or more of the proteins indicates or identifies a disease or condition, a risk of a disease or condition, the severity of a disease or condition, or a combination.

36. The method of claim 28, wherein the absence of one or more of the proteins indicates or identifies a treatment to inhibit, remove or prevent the calcifying nano-particles.

37. The method of claim 28, wherein the absence of one or more of the proteins identifies the type of calcifying nano-particles detected.

38. The method of claim 1, wherein at least one of the proteins is detected using a microarray, coded beads, coated beads, flow cytometry, ELISA, mass spectrometry, fluorescence, chemiluminescence, spectrophotometry, chromatography, electrophoresis, or a combination.

39. The method of claim 1, wherein the proteins on the calcifying nano-particle are detected by
(a) capturing the calcifying nano-particle,
(b) binding a detection compound to one or more of the proteins or components of said particle, and
(c) detecting the detection compound.

40. The method of 39, wherein the calcifying nano-particle is captured by binding a capture compound to one or more of the proteins or components of said particle, wherein the capture compound is or becomes immobilized.

41. The method of claim 40, wherein the proteins to which capture compounds bind mediate capture, wherein the detection compound is bound to one of the proteins or components of said particle, wherein the calcifying nano-particle is characterized by determining which proteins mediate capture of the calcifying nano-particle to which the detected detection compound is bound.

42. The method of claim 40, wherein the capture compound is bound to one of the proteins or components of said particle, wherein the detection compounds detected indicate which of the proteins is present on the calcifying nano-particle, wherein the calcifying nano-particle is characterized by which proteins are present on the calcifying nano-particle.

43. The method of claim 1, wherein the proteins on the calcifying nano-particle are detected by
(a) binding a detection compound to one or more of the proteins,
(b) capturing the calcifying nano-particle, and
(c) detecting the detection compound.

44. A method for detecting one or more proteins, the method comprising
detecting one or more proteins on a calcifying nano-particle.

45. The method of claim 44, wherein the proteins are selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

46. The method of claim 44, wherein two or more proteins are detected on the calcifying nano-particle.

47. The method of claim 44, wherein one or more of the proteins are detected by binding at least one compound to the protein and detecting the bound compound.

48. The method of claim 47, wherein at least one of the compounds is an antibody, wherein the antibody is specific for the protein.

49. The method of claim 44, wherein the calcifying nano-particles comprise calcium phosphate and one or more of the proteins.

50. A method of characterizing a calcifying nano-particle, the method comprising identifying one or more proteins on a calcifying nano-particle.

51. The method of claim 50, wherein the proteins are selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

52. The method of claim 50, wherein the calcifying nano-particles are characterized by identifying two or more proteins on the calcifying nano-particle.

53. The method of claim 50, wherein the identified proteins identify the type of calcifying nano-particle.

54. The method of claim 53, wherein the identified type of calcifying nano-particle is related to or associated with a disease or condition.

55. The method of claim 50, wherein the identified proteins identify a disease or condition with which calcifying nano-particles having the identified proteins are related or associated.

56. The method of claim 50, wherein one or more of the proteins are identified by binding at least one compound to the proteins or components of said particle and detecting the bound compound.

57. The method of claim 56, wherein at least one of the compounds is an antibody, wherein the antibody is specific for the proteins or components of said particle.

58. The method of claim 50, wherein the calcifying nano-particles comprise calcium phosphate and one or more of the proteins or components of said particle.

59. A method of diagnosing a disease or condition, the method comprising
identifying one or more proteins or components on a calcifying nano-particle from a subject, wherein the identified proteins identify a disease or condition with which calcifying nano-particles having the identified proteins are related or associated.

60. The method of claim 59, wherein the proteins are selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

61. The method of claim 59, wherein the disease or condition is diagnosed by identifying two or more proteins or components on the calcifying nano-particle.

62. The method of claim 59, wherein the identified proteins identify a disease or condition that is caused by calcifying nano-particles having the identified proteins or components of said particle.

63. The method of claim 59, wherein the identified proteins identify a disease or condition in which calcifying nano-particles having the identified proteins or components of said particle are produced.

64. The method of claim 59, wherein one or more of the proteins are identified by binding at least one compound to the proteins or components of said particle and detecting the bound proteins or components therein.

65. The method of claim 64, wherein at least one of the compounds is an antibody, wherein the antibody is specific for the protein.

66. The method of claim 59, wherein the calcifying nano-particles comprise calcium phosphate and one or more of the proteins or components of said particle.

67. A method of assessing the prognosis of a disease or condition, the method comprising
identifying one or more proteins or components on a calcifying nano-particle from a subject, wherein the identified proteins identify calcifying nano-particles that are related to or associated with the prognosis of the disease or condition.

68. The method of claim 67, wherein the proteins are selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

69. The method of claim 67, wherein the prognosis of a disease or condition is assessed by identifying two or more proteins or components on the calcifying nano-particle.

70. The method of claim 67, wherein one or more of the proteins or components of said particle are identified by binding at least one compound to said proteins or components and detecting the bound compound.

71. The method of claim 70, wherein at least one of the compounds is an antibody, wherein the antibody is specific for the proteins or components of said particle.

72. The method of claim 67, wherein the calcifying nano-particles comprise calcium phosphate and one or more of the proteins or components of said particle.

73. A method of identifying a subject at risk of a disease or condition, the method comprising identifying one or more proteins or components on a calcifying nano-particle from a subject, wherein the identified proteins or components identify calcifying nano-particles that are related to or associated with a risk of developing a disease or condition.

74. The method of claim 73, wherein the proteins are selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

75. The method of claim 73, wherein the subject is identified by identifying two or more proteins on the calcifying nano-particle.

76. The method of claim 73, wherein one or more of the proteins or components are identified by binding at least one compound to the proteins or component and detecting the bound compound.

77. The method of claim 76, wherein at least one of the compounds is an antibody, wherein the antibody is specific for the protein or component.

78. The method of claim 73, wherein the calcifying nano-particles comprise calcium phosphate and one or more of the protein or components of said particle.

79. An isolated calcifying nano-particle, wherein the calcifying nano-particle comprises one or more of the proteins selected from the group consisting of proteins anti-Fetuin A, calmodulin, Tgase II, MMP-9, MMP-3, CD 42b, NF-kappa B, osteopontin, Factor X/Xa, CD14, prothrombine, Factor IX, Fetuin B, CD40, anti-myeloperoxidase, Fibronectin, Factor VII, tissue factor, human complement 5b-9, human CRP, matrix GLA, CD61, Kappa Light Chain, Macrophage, factor XIIIA, hsp 60, fibrillin-1, B2 microgl, CD 18, laminin, trypsin, Notch-1, BSA, LBP, PTX3, complement C5, fibrinogen, D-Dimer, factor V, human gamma-G1a, TF-VIIa, complement C3c, Complement C4, antichymotrypsin, Annexin V, Lipid A, isopeptide bond, vitronectin, thrombin, osteocalcin, Troponin T, vimentin, tropomyosin, HAS, Troponin I cardiac, Apo A1, MHC class I, Amyloid P protein, sCD40 L, kallikrein, Prothr F1, goat-ATIII, Thrombin, Factor VIII, heparan Sulph, Factor XI, c-jun, Fra-2, Fra-1, Jun B, P-c-Jun, TGase3, alpha fetoprotein, PSA, erbB2, VEGF, alpha synuclein, mucin-1, Cystatin A, Cystatin S, Prostein, Aquaporin 4, Trypsin, Osteonectin, RAGE, PGRP-1 Beeta, PGRP-S, Gram positive bacteria, Troponin C Cardiac, Protein C, Macrophage Scavenger Receptor Type I, anti-Thrombin, Protein S, BAFF on the calcifying nano-particle.

80. A composition comprising a calcifying nano-particle and one or more compounds bound to one or more proteins or components on the calcifying nano-particle.

81. The composition of claim 80, wherein the composition comprises a calcifying nano-particle and one or more compounds bound to two or more proteins or components on the calcifying nano-particle.

82. The composition of claim 80, wherein at least one of the compounds is an antibody, wherein the antibody is specific for the protein or components.

83. The composition of claim 80, wherein the calcifying nano-particles comprise calcium phosphate and one or more of the proteins or components.

84. The composition of claim 80, wherein at least one of the compounds blocks the calcifying nano-particle.

85. A method of detecting a particle wherein proteins on the particle are detected by
(a) capturing the particle,
(b) binding a detection compound to one or more of the proteins or components of said particle, and
(c) detecting the detection compound.

86. The method of claim 85, wherein said particle is a stable particle, such as a microparticle, virus, spore, bacteria, prion, mineral, metal, or synthetic particle as introduced into the circulation of an animal.

87. The method of claim 85 wherein said proteins or compounds are quantitated using a single standard curve.

88. The method of claim 87, wherein said curve is created by including, as the standard, at least one protein or other component of said particle as a standard for the assay

89. The method of claim 87, wherein said curve is created by including various concentrations of CNPs or at least one of the CNP antigen into the assay format.

90. The method of claim 1, wherein said proteins or components may be any of those that adhere to the surface of said particle.

91. The method of claim 1, wherein said proteins may be any calcium binding protein.

92. The method of claim 1, wherein said proteins may be any proteins that bind to calcium binding proteins.

93. The method of claim 39, wherein said detector antibody is directed against conformationally changed or chemically modified epitope.

94. The method of claim 39, wherein said proteins or compounds are quantitated using a single standard curve.

95. The method of claim 39, wherein said curve is created by including various concentrations of CNPs or at least one of the CNP antigen into the assay format.

96. The method of claim 39, wherein said proteins or components may be any of those that adhere to the surface of said particle.

97. The method of claim 39, wherein said proteins may be any calcium binding protein.

98. The method of claim 39, wherein said proteins may be any proteins that bind to calcium binding proteins.

99. A kit comprising one or more detection compounds, one or more capture compounds, and one or more solid supports for the detection of calcifying nanoparticles and assessment or quantification of the proteins or components associated thereupon.

100. The method of claim 59, wherein said pattern of said proteins indicate or identify a disease or condition, or a combination of said diseases or conditions including but not limited to heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

101. The method of claim 67, wherein said pattern of said proteins indicate prognosis of a disease or condition including, but not limited to heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

102. The method of claim 73, wherein said pattern of said proteins indicates risk of a disease or condition, or combination of diseases or conditions, including but not limited to heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts, Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

103. A method of identifying a treatment to inhibit, remove or prevent the calcifying nano-particles or monitor response to said treatment, the method comprising identifying one or more proteins or components on a calcifying nano-particle from a subject, wherein the identified proteins are related to or associated with the selection of therapy or for predicting response to treatment.

104. A method for detecting calcifying nanoparticles on or in foreign devices implanted or to be implanted, or introduced into body cavities including, but not limited to stents, scopes, tubes, endoscopes, catheters, pumps, pace makers, dental appliances, and other implants by detecting one ore more proteins or components on the calcifying nano-particle.

105. A method for detecting calcifying nano-particles in biological materials or donors thereof including, but not limited to blood, tissues, organs, cells, and biopharmaceutical products by detecting one ore more proteins or components on the calcifying nano-particle.

106. A method for detecting calcifying nano-particles in biological materials or donors thereof including but not limited to dairy products, meats, water, and other food stuffs by detecting one or more proteins or components on the calcifying nano-particle.

107. A method for determining risk of future severe adverse health events for individuals to be placed in extreme, demanding, or solitary environments including, but not limited to astronauts, military personnel, and explorers or for aiding in the determination of insurance risk by detecting one ore more proteins or components, or patterns thereof, on the calcifying nano-particle.
